(19) United States
(12) Patent Application Publication
(10) Pub. No.: US 2005/0209256 A1
(43) Pub. Date: Sep. 22, 2005

(54) Novel beta-turn mimetics as calcitonin gene related peptide receptor antagonists

(76) Inventors: Charles J. Andres JR., Wethersfield, CT (US); Paul Michael Scola, Glastonbury, CT (US); Katharine A. Grant-Young, Madison, CT (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/083,271

(22) Filed: Mar. 17, 2005

Related U.S. Application Data
(60) Provisional application No. 60/554,626, filed on Mar. 19, 2004.
(60) Provisional application No. 60/554,646, filed on Mar. 19, 2004.

(51) Int. Cl. A61K 31/519; C07D 487/02

(52) U.S. Cl. 514/267; 544/255

(57) ABSTRACT

Conformationally constrained compounds which mimic the secondary structure of reverse-turn regions of biologically active peptides are disclosed. The compounds of the present invention of Formula I have valuable pharmacological properties based on their ability to selectively antagonize calcitonin gene-related peptide (CGRP) receptor for acute and prophylactic treatment of headaches, particularly migraines.

### CROSS-REFERENCE TO RELATED APPLICATIONS

This is a non-provisional application which claims the benefit of U.S. Provisional Application No. 60/554,626 filed Mar. 19, 2004.

### FIELD OF THE INVENTION

This invention relates to a method of treatment of migraine headaches. More particularly, it relates to the treatment of migraine headaches with agents that inhibit the action or actions of calcitonin gene related peptide (CGRP).

### BACKGROUND OF THE INVENTION

Migraine headaches are one of the most prevailing neurological disorders with recurring attack of pain lasting between 4 to 72 hours. Migraines are known to produce the most intense headaches reported. Migraines involve a plethora of symptoms including predominantly unilateral dull pain at the beginning and then pulsing headaches occur with moderate to severe intensity. Symptoms that typically accompany migraines include hypersensitivity towards light and sound, pallor, and mild to extreme nausea and vomiting.

There are varying ideas on the pathophysiology of the migraine. A variety of stress stimuli, including intense light, noise, anxiety, exertion, extremes of temperature, hormones, exhaustion, infection and trauma result in constriction of extracranial blood vessels. The vasoconstriction is followed by reflexive vasodilation that subsequently spreads to intracranial vessels. Increased levels of norepinephrine, serotonin, histamine, and various neuoropeptides are considered to be the main endogenous pain producing molecules, accompanied by direct sensory nerve stimulation because of the stretching that accompanies vasoconstriction and dilation.

Other theories suggest that the accompanying headaches are triggered not only by vasodilation, but also by a central lowering of the pain threshold with immediate early genes (IEGs) being activated in the cells of the spinal cord and of the brain stem after stimulation.

A different theory—the neurogenic inflammation model—offers a possibility of explaining the blood flow change as well as the increased pain sensitivity of the vessels during migraine attacks. According to this theory, the increased pain sensitivity is brought about by an increased sensitization of the sensory perivascular fibers of the trigerminovascular system. Vascular pulsations, which normally are not capable of initiating painful sensations are potent pain stimuli due to this increased sensitization, and bring about the pulsing, throbbing migraine. Several observations point to the involvement of neuropeptides, such as bradykinin, substance P, and in particular, calcitonin gene-related peptide (CGRP). In the brain, the calcitonin gene transcript is spliced to produce an mRNA which encodes CGRP. CGRP is a 37-amino acid neuropeptide which is the most potent naturally occurring vasodilator in the human body. CGRP occurs naturally in either the alpha or beta form and is distributed throughout the central and peripheral nervous system.

Migraine headaches involve the activation of the trigeminal system and dilation of cranial vessels. CGRP is concentrated in neurons in the trigeminal ganglia and increased CGRP levels can be detected during a migraine attack, presumably causing the vasodilation observed. During a migraine attack, CGRP can also be detected in increased amounts in the venous blood of the head. Accordingly, a possible treatment for migraine attacks may involve inhibition of CGRP provoked dilation of the cranial vessels.

The known antimigraine drugs include ergot alkaloids and the so-called “triptans”, e.g. sumatriptan and zolmitriptan. These drugs have vasoconstrictive properties and presumably inhibit the release of neuropeptide CGRP (Ferrari, M. D., Saxena, P. R. Eur. J. Neurology 2: 5-21 (1995). Thus, a novel approach to treat migraine is the use of CGRP antagonists as described by Doods, H., et al, in Pharmacological profile of BIBN4096BS, the first selective small molecule CGRP antagonist, Br. J. Pharmacol., 129, 420-423 (2000). Despite these examples there remains a need for novel and potent CGRP receptor antagonists. According to the present invention various beta-turn mimetics were discovered to be potent CGRP antagonists. Other beta-turn mimetics are described in Eguchi, Masakatsu; Lee, Min S.; Nakanishi, Hiroshi; Stasiak, Marcin; Lovell, Scott; Kahn, Michael. Solid-phase synthesis and structural analysis of bicyclic b-turn mimetics incorporating functionality at the i to i+3 positions. Journal of the American Chemical Society (1999), 121(51), 12204-12205. CODEN: JACSAT ISSN:0002-7863. CAN 132:222508 AN 1999:787550 CAPLUS; Eguchi, Masakatsu; Shen, Richard Y. W.; Shea, J. Paul; Lee, Min S.; Kahn, Michael. Design, Synthesis, and Evaluation of Opioid Analogues with Non-Peptidic b-Turn Scaffold: Enkephalin and Endomorphin Mimetics. Journal of Medicinal Chemistry (2002), 45(7), 1395-1398. CODEN: JMCMAR ISSN:0022-2623. CAN 136:350114 AN 2002:159133 CAPLUS; Kahn, Michael S.; Eguchi, Masakatsu; Kim, Hwa-ok. Preparation of conformationally constrained bicyclic peptide derivatives as reverse-turn mimetics. PCT Int. Appl. (1998), 68 pp. CODEN: PIXXD2 WO 9849168 A1 19981105 CAN 129:343724 AN 1998:721701 CAPLUS; U.S. Pat. No. 6,013,458 and U.S. Pat. No. 5,929,237. None of these references show that beta-turn mimetics are useful as CGRP receptor antagonists. Thus the present invention embodies a significant contribution by discovering various beta-turn mimetics as CGRP receptor antagonists useful for the treatment of migraine.

### SUMMARY OF THE INVENTION

The according to a first aspect of the present invention are provided compounds of Formula I and pharmaceutically acceptable salts and solvates thereof wherein 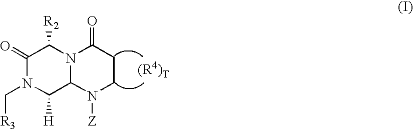
Z is CONHR₁ or CO₂R₁ wherein
   R₁ is phenyl substituted in the para position with a ketone or isostere thereof or an alcohol or isostere thereof;
R₂ is
   C₁-C₄ alkyl, C₃-C₈ cycloalkyl, acetic acid adamantan-2-yl ester, acetic acid benzyl ester, butyl carbamic acid benzyl ester, benzyloxy methyl, 4-benzyloxy benzyl, piperidin-4-yl, tetrahydro-pyran-4-yl, 3-methyl-3H-imidazol-4-yl methyl, 2-carbamoyl-ethyl, C₁-C₄ alkyl S(O)ₙ C₁-C₄ alkyl wherein n is 0, 1, or 2,
      benzyl or phenyl, wherein either or both of said benzyl or phenyl are optionally substituted on the aryl moiety with one or more of the same or different substituents selected from the group consisting of
         S(O)ₘ C₁-C₄ alkyl wherein m is 0, 1 or 2;
         nitro;
         hydroxy;
         fluoro; and
         chloro;
R₃ is
   benzyl optionally substituted in the meta position with fluoro or chloro,
   2-pyridin-4-yl-methyl, 2-thiophen-2-yl methyl or 2-pyridin-3-yl-methyl;
R₄ together with the carbon atoms to which it is attached is phenyl or napthyl; and
T is 0 or 1.

The according to another embodiment of the first aspect of the present invention are provided compounds of Formula II and pharmaceutically acceptable salts and solvates thereof wherein 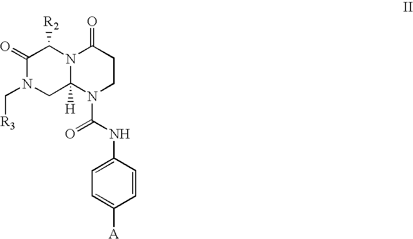
A is a ketone or isostere thereof or an alcohol or isostere thereof R₂ is
   C₁-C₄ alkyl, C₃-C₈ cycloalkyl, acetic acid adamantan-2-yl ester, acetic acid benzyl ester, butyl carbamic acid benzyl ester, benzyloxy methyl, 4-benzyloxy benzyl, piperidin-4-yl, tetrahydro-pyran-4-yl, 3-methyl-3H-imidazol-4-yl methyl, 2-carbamoyl-ethyl, C₁-C₄ alkyl S(O)ₙ C₁-C₄ alkyl wherein n is 0, 1, or 2,
   benzyl or phenyl, wherein either or both of said benzyl or phenyl are optionally substituted on the aryl portion moiety with one or more of the same or different substituents selected from the group consisting of
      S(O)ₘ C₁-C₄ alkyl wherein m is 0, 1 or 2;
      nitro;
      hydroxy;
      fluoro; and
      chloro;
R₃ is
   benzyl optionally substituted in the meta position with fluoro or chloro,
   2-pyridin-4-yl-methyl, 2-thiophen-2-yl methyl or 2-pyridin-3-yl-methyl.

The according to another embodiment of the first aspect of the present invention are provided compounds of Formula III and pharmaceutically acceptable salts and solvates thereof wherein 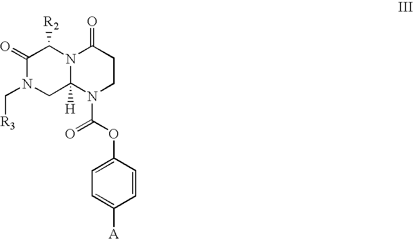
A is a ketone or isostere thereof or an alcohol or isostere thereof;
R₂ is
   C₁-C₄ alkyl, C₃-C₈ cycloalkyl, acetic acid adamantan-2-yl ester, acetic acid benzyl ester, butyl carbamic acid benzyl ester, benzyloxy methyl, 4-benzyloxy benzyl, piperidin-4-yl, tetrahydro-pyran-4-yl, 3-methyl-3H-imidazol-4-yl methyl, 2-carbamoyl-ethyl, C₁-C₄ alkyl S(O)ₙ C₁-C₄ alkyl wherein n is 0, 1, or 2,
   benzyl or phenyl, wherein either or both of said benzyl or phenyl are optionally substituted on the aryl portion moiety with one or more of the same or different substituents selected from the group consisting of
      S(O)ₘ C₁-C₄ alkyl wherein m is 0, 1 or 2;
      nitro;
      hydroxy;
      fluoro; and
      chloro;
R₃ is
   benzyl optionally substituted in the meta position with fluoro or chloro,
   2-pyridin-4-yl-methyl, 2-thiophen-2-yl methyl or 2-pyridin-3-yl-methyl.

The according to another embodiment of the first aspect of the present invention are provided compounds of Formula IV and pharmaceutically acceptable salts and solvates thereof wherein 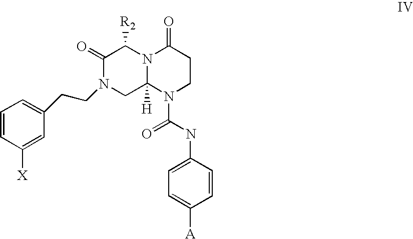
X is H or F;
A is a ketone or isostere thereof or an alcohol or isostere thereof; and
R₂ is
   C₁-C₄ alkyl, C₃-C₈ cycloalkyl, acetic acid adamantan-2-yl ester, acetic acid benzyl ester, butyl carbamic acid benzyl ester, benzyloxy methyl, 4-benzyloxy benzyl, piperidin-4-yl, tetrahydro-pyran-4-yl, 3-methyl-3H-imidazol-4-yl methyl, 2-carbamoyl-ethyl, C₁-C₄ alkyl S(O)ₙ C₁-C₄ alkyl wherein n is 0, 1, or 2,
   benzyl or phenyl, wherein either or both of said benzyl or phenyl are optionally substituted on the aryl portion moiety with one or more of the same or different substituents selected from the group consisting of
      S(O)ₘ C₁-C₄ alkyl wherein m is 0, 1 or 2;
      nitro;
      hydroxy;
      fluoro; and
      chloro.

Embodiments of a second aspect of the present invention provide a method of treating a subject afflicted with migraine headaches comprising administering to said subject a pharmaceutically effective amount of a compound of the present invention as defined herein in a pharmaceutical preparation.

Embodiments of a third aspect of the present invention provide a method of treating a subject afflicted with migraine headaches, wherein said migraine headaches are characterized by overexpression of CGRP, comprising administering to said subject a pharmaceutically effective amount of a compound of the present invention as defined herein in a pharmaceutical preparation.

Embodiments of a fourth aspect of the present invention provide a method of treating a subject afflicted with migraine headaches comprising administering to said subject a composition comprising a pharmaceutically effective amount of a compound of the present invention as defined herein in a pharmaceutical preparation in combination with an antiemetic.

Embodiments of a fifth aspect of the present invention provide a method for treating a subject afflicted with a disease characterized by excessive vasodilatation and consequent reduction in blood flow, comprising administering to said subject a composition comprising a pharmaceutically effective amount of a compound of the present invention as defined herein.

### DETAILED DESCRIPTION OF THE INVENTION

The compounds of the present invention have valuable pharmacological properties based on their ability to selectively antagonize calcitonin gene related peptide (CGRP) receptors. The invention further relates to pharmaceutical compositions containing these compounds, their use in the treatment of migraine and the preparation thereof.

The description of the invention herein should be construed in congruity with the law and principles of chemical bonding. Where a variable is defined as having the value of zero, it is understood that the bond attached to said variable should be removed. The numbers in the subscript after the symbol “C” define the number of carbon atoms a particular group can contain. For example “C₁-C₄ alkyl” means saturated hydrogen chains, branched or unbranched having one to four carbon atoms, including without limitation groups such as methyl, ethyl, n-propyl, isopropyl, methylpropyl, n-butyl, t-butyl, isobutyl and sec-butyl.

“Cycloalkyl” means saturated ring systems, including mono-, bi-, or polycyclic ring systems. Cycloalkyl includes without limitation cyclopropyl, cyclobutyl, cyclopentyl, cyclohexyl, cyclohexylmethyl and the like.

“Ketone Isostere” means any moiety having the functional equivalent activity of a ketone and includes but is not limited to, CO, SO, SO₂, S, CNR, CNN(H)R, P(O)OH, or P(O)OR where R is H, C₁₋₆ alkyl or aryl. “Alcohol isostere” means any moiety having the functional equivalent activity of an alcohol and includes but is not limited to CH₂OH, CH₂SH, POH, CH₂NH₂, or CH₂NHR.

In addition to the compounds described and listed hereinabove, this invention provides their corresponding pharmaceutically acceptable salt, hydrate, solvate, radiolabelled, various stereoisomeric and prodrug forms. “Pharmaceutically acceptable salts” of compounds of this invention are also provided herein. The phrase “pharmaceutically acceptable” is employed to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, or alkali or organic salts of acidic residues such as carboxylic acids.

Pharmaceutically acceptable salts include the conventional non-toxic salts or the quaternary ammonium salts of the parent compound formed, for example, from non-toxic inorganic or organic acids. Such conventional nontoxic salts include those derived from inorganic acids such as hydrochloric, hydrobromic, sulfuric, sulfamic phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

Pharmaceutically acceptable salt forms of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, the disclosure of which is hereby incorporated by reference.

Radiolabelled compounds, i.e. wherein one or more of the atoms described are replaced by a radioactive isotope of that atom (e.g. C replaced by 14 C or by 11C, and H replaced by 3H or 18F), are also provided for herein. Such compounds have a variety of potential uses, e.g. as standards and reagents in determining the ability of a potential pharmaceutical to bind to neurotransmitter proteins, or for imaging compounds of this invention bound to biological receptors in vivo or in vitro.

Each of the steroisomeric forms of this invention's compounds is also provided for herein. That is, the compounds can have one or more asymmetric centers or planes, and all chiral (enantiomeric and diastereomeric) and recemic forms of the compounds are included in the present invention. Many geometric isomers of olefins, C═N double bonds, and the like can also be present in the compounds, and all such stable isomers are contemplated in the present invention. Compounds are synthesized and isolated the optically pure form.

Prodrug forms of this invention's compounds are also provided for herein. Such “prodrugs” are compounds comprising this invention's compounds and moieties covalently bound to the parent compounds such that the portions of the parent compound most likely to be involved with toxicities in subjects to which the prodrugs have been administered are blocked from inducing such effects. However, the prodrugs are also cleaved in the subjects in such a way as to release the parent compound without unduly lessening its therapeutic potential. Prodrugs include compounds wherein hydroxy, amine, or sulfhydryl groups are bonded to any group that, when administered to a mammalian subject, cleaves to form a free hydroxyl, amino, or sulfhydryl group, respectively. Examples of prodrugs include, but are not limited to, acetate, formate, and benzoate derivatives of alcohol, and amine functional groups in the compounds of Formulae (I-III).

The compounds provided herein are, for example and without limitation, made by the synthetic routes and schemes set forth hereinbelow.

Moreover, in addition to compounds made by these routes and schemes, this invention provides pharmaceutical compositions comprising pharmaceutically acceptable carriers and therapeutically effective amounts of the compounds. “Pharmaceutically acceptable carriers” are media generally accepted in the art for the delivery of biologically active agents to animals, in particular, mammals. Such media are formulated according to a number of factors well within the purview of those of ordinary skill in the art to determine and account for. These include, without limitation: the type and nature of the active agent being formulated; the subject to which the agent-containing composition is to be administered; the intended route of administration of the composition; and, the therapeutic indication being targeted.

Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Descriptions of suitable pharmaceutically acceptable carriers, and factors involved in their selection, are found in a variety of readily available sources, e.g., Remington's.

This invention thus further provides a method of treating a subject afflicted with a neurological disorder characterized by CGRP overexpression, such as migraine headaches as described hereinabove, which comprises administering to the subject a pharmaceutical composition provided herein. Such compositions generally comprise a therapeutically effective amount of a compound provided herein, that is, an amount effective to ameliorate, lessen or inhibit disorders characterized by CGRP overexpression. Such amounts typically comprise from about 0.1 to about 1000 mg of the compound per kg of body weight of the subject to which the composition is administered. Therapeutically effective amounts can be administered according to any dosing regimen satisfactory to those of ordinary skill in the art.

Administration is, for example, by various parenteral means. Pharmaceutical compositions suitable for parenteral administration include various aqueous media such as aqueous dextrose and saline solutions; glycol solutions are also useful carriers, and preferably contain a water soluble salt of the active ingredient, suitable stabilizing agents, and if necessary, buffer substances. Antioxidizing agents, such as sodium bisulfite, sodium sulfite, or ascorbic acid, either alone or in combination, are suitable stabilizing agents; also used are citric acid and its salts, and EDTA. In addition, parental solutions can contain preservatives such as benzalkonium chloride, methyl- or propyl-paraben, and chlorobutanol.

Alternatively, compositions can be administered orally in solid dosage forms, such as capsules, tablets and powders; or in liquid forms such as elixirs, syrups, and/or suspensions Gelatin capsules can be used to contain the active ingredient and a suitable carrier such as but not limited to lactose, starch, magnesium stearate, stearic acid, or cellulose derivatives. Similar diluents can be used to make compressed tablets. Both tablets and capsules can be manufactured as sustained release products to provide for continuous release of medication over a period of time. Compressed tablets can be sugar-coated or film-coated to make any unpleasant taste, or used to protect the active ingredients from the atmosphere, or to allow selective disintegration of the tablet in the gastrointestinal tract.

This invention is described in the following examples, which those of ordinary skill in the art will readily understand are not limiting on the invention as defined in the claims, which follow thereafter.

### Synthesis

The hexahydro-pyrazino[1,2-a]pyrimidines of the present invention may be prepared using the procedures outlined in Scheme 1 and exemplified by the solid phase synthesis of (6S,9aS)-6-Cyclohexyl-4,7-dioxo-8-phenethyl-hexahydro-pyrazino[1,2-a]pyrimidine-1-carboxylic acid (4-acetyl-phenyl)-amide 1 as shown below.

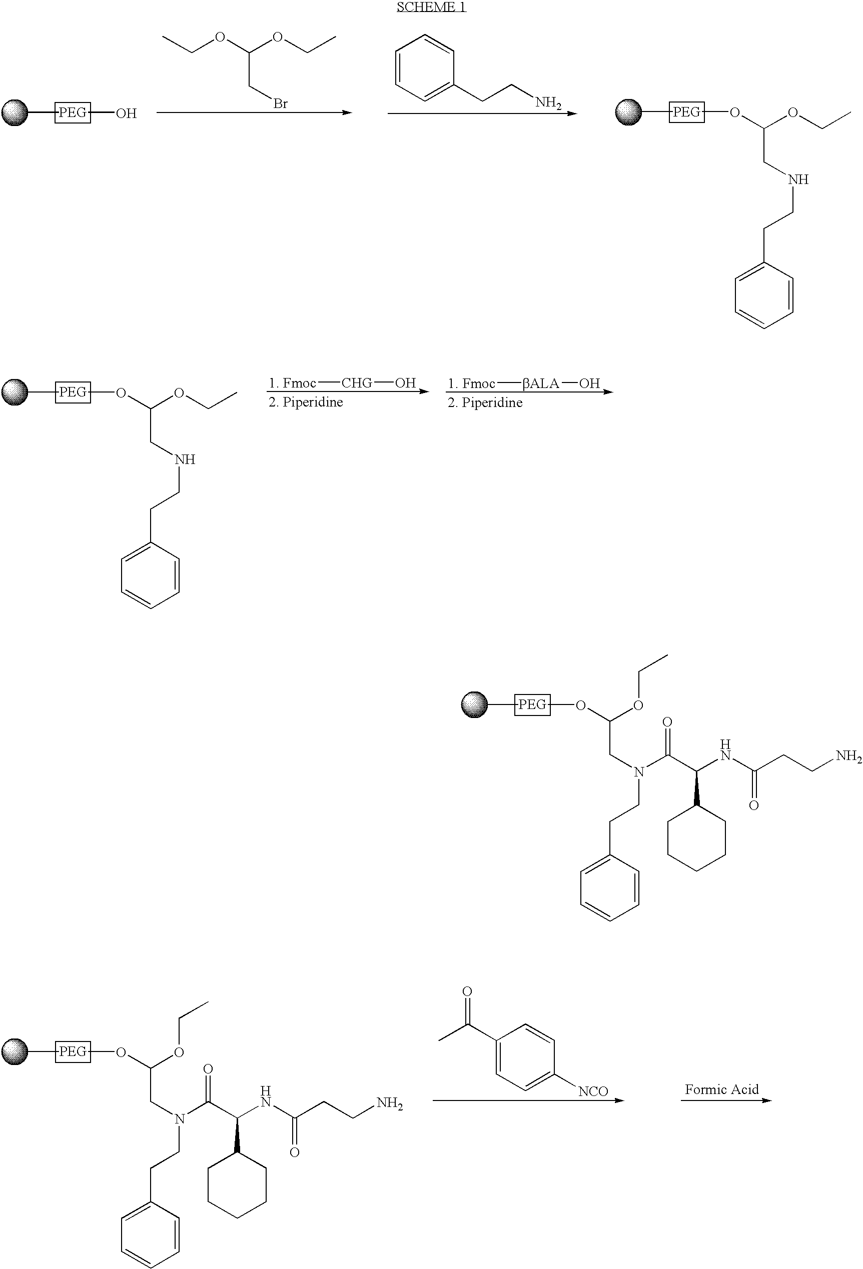

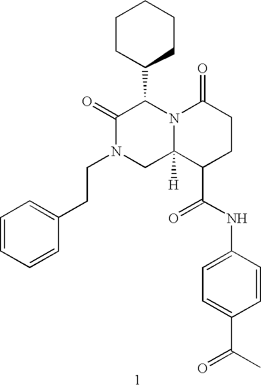

To a 500 mL round bottom flask was added 10.0 g of PEG-grafted Polystyrene hydroxyl resin (0.48 mmol/g), 3.74 g (14.9 mmol) of PPTS (pyridinium p-toluenesulfonate) and 200 mL of dry DCE (1,2-dichloroethane) under Argon. The round bottom flask was fitted with a short-path distillation column and 50 mL of DCE was distilled off at one atmosphere. To the reaction mixture was then added 9.0 mL of bromoacetaldehyde diethyl acetal (11.8 g, 59.8 mmol) in 50 mL of DCE. The short path distillation column was employed again to remove 50 mL of DCE. The addition of the acetal and distillation procedure was repeated. Upon cooling, the resin was filtered, washed three times with DMF (N,N-dimethylformamide) and Dioxane.

To the resulting resin was added a 0.2M solution of Phenethylamine in DMSO (Dimethylsulfoxide). The resulting mixture was shaken at 60° C. over night. Upon return to room temperature, the resin was drained, washed five times with DMF and DCM (Dichloromethane).

To the phenethylamine resin was added a 0.2M solution of DIC (Diisopropylcarbodiimide), HOAT (1-Hydroxy-7-azabenzotriazole), and FMOC-CHG-OH (N-α-Fluorenylmethoxycarbonyl-β-cyclohexyl-L-glycine). The resulting mixture was shaken at room temperature over night. The resin was drained and washed five times with DMF and DCM.

To the Fmoc-cyclohexylglycine resin was added a 20% piperidine solution in DMF. The mixture was shaken for 30 minutes at room temperature then drained, and washed five times with DMF and DCM. To the deprotected amine resin was added a 0.2M solution of DIC (Diisopropylcarbodiimide), HOBT (N-Hydroxybenzotriazole), and FMOC-β-Ala-OH (N-α-Fluorenylmethoxycarbonyl-β-alanine). The mixture was shaken overnight at room temperature. The resulting resin mixture was drained and washed five times with DMF and DCM.

To the Fmoc-β-alanine resin was added a 20% piperidine solution in DMF. The mixture was shaken for a half-hour at room temperature then drained, and washed five times with DMF and DCM. To the β-alanine resin was added a 0.2M solution of 4-Acetylphenyl isocyanate in DCE. The mixture was shaken over night at room temperature. The resulting resin mixture was drained, then washed several times with DMF and DCM.

Addition of formic acid to the resulting resin followed by shaking at room temperature over night afforded (6S,9aS)-6-Cyclohexyl-4,7-dioxo-8-phenethyl-hexahydro-pyrazino[1,2-a]pyrimidine-1-carboxylic acid (4-acetyl-phenyl)-amide in a 24% yield, as a white solid.

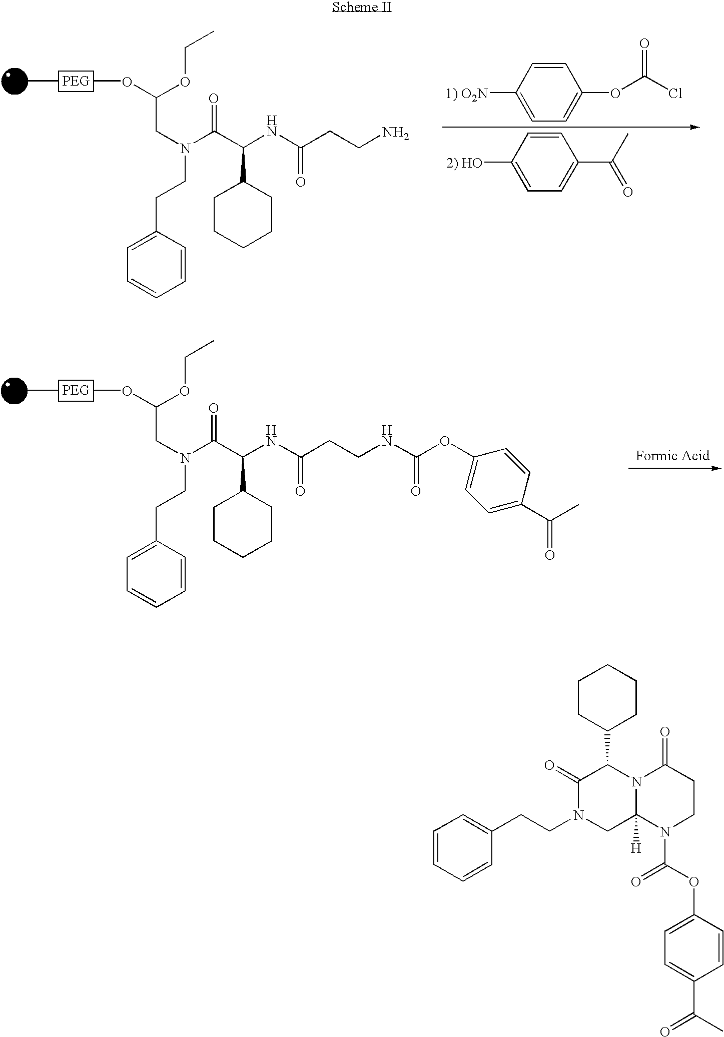

The intermediate amine derived from steps shown in SCHEME 1 is treated with p-nitro phenol chloroformate and triethylamine in a 1:1 dichloromethane:tetrahydorfuran solvent mixture as described in Angew Chem Int. Ed. Engl. (1995), 34, 907-909. After shaking for one half hour, the solvent is removed by filtration, the resin washed with 1:1 dichloromethane:tetrahydrofuran, and a solution of alcohol or phenol and triethylamine in N,N-dimethylformamide is added. After shaking for four hours, the alcohol or phenol solution is removed by filtration and the resin is washed sequentially with N,N-dimethylformamide, dichloromethane, and tetrahydrofuran. The resin is vacuum dried and treated with formic acid for four to fourteen hours to afford product 149.

### EXAMPLES

Analytical data were recorded for the compounds described below using the following general procedures. All NMR spectra were recorded at room temperature using a Varian XL-300, a Bruker DPX-300, or a Bruker DRX500 spectrometer. The NMR solvents used were deuterochloroform (CDCl₃) and methyl alcohol-d₄ (CD₃OD). Chemical shifts are reported in ppm relative to CDCl₃ or CD₃OD. Coupling constants were reported in hertz. Peak multiplicity was reported using the following abbreviations: s (singlet), d (doublet), t (triplet), m (multiplet), br (broad), dd (doublet of doublets), td (triplet of doublets).

LC/MS data was obtained on a Shimadzu LC-10AS and a Micromass Platform LC (ESI+) at 220 nm using the following set of conditions:

(Method A) YMC XTerra 71 μm C18, 3.0×50 mm column, or a YMC XTerra 5 μm C18, 4.6×50 mm column, at 5 mL/min, with a linear gradient of 0-100% B (B=90% HPLC grade methanol, 10% HPLC grade water and 0.1% trifluoroacetic acid), in 2 minutes with a 1 minute hold.

(Method B) YMC ODS C18, 7 μm, 3.0×50 mm column, at 4 mL/min, with a linear gradient of 0-100% B (B=90% HPLC grade methanol, 10% HPLC grade water and 0.1% trifluoroacetic acid), in 4 minutes with a 1 minute hold.

Chiral HPLC data was recorded at 220 nm using a Chiralcel OD 10 μm 4.6×250 mm column with 75% HPLC grade ethanol/25% HPLC grade hexane, at 1.0 mL/min for 40 minutes.

Reagents were purchased from commercial sources and were used without further purification.

Table I is a brief summary of compounds provided herein made according to the synthetic schemes described above and the examples provided below.

TABLE 1

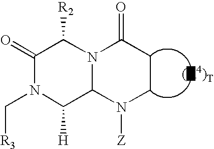

| Example No. | Z | | R₂ | R₃ | T, R₄ |
|---|---|---|---|---|---|
| | CONHR₁ | COOR₁ | | | 0, N/A |
| 1 | 4-acetyl phenyl | | Cyclohexyl | Benzyl | 0, N/A |
| 2 | 4-methane sulfonyl-phenyl | | Cyclohexyl | Benzyl | 0, N/A |
| 3 | 4-methanesulfinyl-phenyl | | Cyclohexyl | Benzyl | 0, N/A |
| 4 | 4-methylsulfanyl-phenyl | | Cyclohexyl | Benzyl | 0, N/A |
| 5 | 4-acetyl phenyl | | Isopropyl | 2-(3-fluoro-benzyl) | 0, N/A |
| 6 | 4-acetyl-phenyl | | 1S-Methylpropyl | 2-(3-fluoro-benzyl) | 0, N/A |
| 7 | 4-acetyl-phenyl | | Benzyl | 2-(3-fluoro-benzyl) | 0, N/A |
| 8 | 4-acetyl-phenyl | | Isobutyl | 2-(3-fluoro-benzyl) | 0, N/A |
| 9 | 4-acetyl-phenyl | | 2-methyl sulfanyl-ethyl | 2-(3-fluoro-benzyl) | 0, N/A |
| 10 | 4-acetyl-phenyl | | Methyl | 2-(3-fluoro-benzyl) | 0, N/A |
| 11 | 4-acetyl-phenyl | | Butyl | 2-(3-fluoro-benzyl) | 0, N/A |
| 12 | 4-acetyl-phenyl | | 4-nitro benzyl | 2-(3-fluoro-benzyl) | 0, N/A |
| 13 | 4-acetyl-phenyl | | Cyclohexylmethyl | 2-(3-fluoro-benzyl) | 0, N/A |
| 14 | 4-acetyl-phenyl | | acetic acid | 2-(3-fluoro-benzyl) | 0, N/A |
| | | | adamantan-2-yl ester | | |
| 15 | 4-acetyl-phenyl | | Acetic acid benzyl ester | 2-(3-fluoro-benzyl) | 0, N/A |
| 16 | 4-acetyl-phenyl | | Butyl carbamic acid benzyl ester | 2-(3-fluoro-benzyl) | 0, N/A |
| 17 | 4-acetyl-phenyl | | Benzyloxy methyl | 2-(3-fluoro-benzyl) | 0, N/A |
| 18 | 4-acetyl-phenyl | | 4-Benzyloxy benzyl | 2-(3-fluoro-benzyl) | 0, N/A |
| 19 | 4-acetyl-phenyl | | 3,5-dibromo-4-hydroxy-benzyl | 2-(3-fluoro-benzyl) | 0, N/A |
| 20 | 4-acetyl-phenyl | | Ethyl sulfanyl methyl | 2-(3-fluoro-benzyl) | 0, N/A |
| 21 | 4-acetyl-phenyl | | 2-methane sulfonyl ether | 2-(3-fluoro-benzyl) | 0, N/A |
| 22 | 4-acetyl-phenyl | | Methyl sulfanyl methyl | 2-(3-fluoro-benzyl) | 0, N/A |
| 23 | 4-acetyl-phenyl | | 1R-Methylpropyl | 2-(3-fluoro-benzyl) | 0, N/A |
| 24 | 4-acetyl-phenyl | | 2-carbamoyl-ethyl | 2-(3-fluoro-benzyl) | 0, N/A |
| 25 | 4-acetyl-phenyl | | Benzyl sulfanyl methyl | 2-(3-fluoro-benzyl) | 0, N/A |
| 26 | 4-acetyl-phenyl | | 2-methane sulfinyl-ethyl | 2-(3-fluoro-benzyl) | 0, N/A |
| 27 | 4-acetyl-phenyl | | Piperidin-4-yl | 2-(3-fluoro-benzyl) | 0, N/A |
| 28 | 4-acetyl-phenyl | | Phenylmethanesulfinylmethyl | 2-(3-fluoro-benzyl) | 0, N/A |
| 29 | 4-acetyl-phenyl | | Phenylmethanesulfonylmethyl | 2-(3-fluoro-benzyl) | 0, N/A |
| 30 | 4-acetyl-phenyl | | phenyl | 2-(3-fluoro-benzyl) | 0, N/A |
| 31 | 4-acetyl-phenyl | | 1R-methylpropyl | 2-(3-fluoro-benzyl) | 0, N/A |
| 32 | 4-acetyl-phenyl | | Cyclohexyl | 2-(3-fluoro-benzyl) | 0, N/A |
| 33 | 4-acetyl-phenyl | | Tetrahydro-pyran-4-yl | 2-(3-fluoro-benzyl | 0, N/A |
| 34 | 4-acetyl-phenyl | | Tetrahydro-pyran-4-yl | 2-(3-fluoro-benzyl) | 1, naphthyl |
| 35 | 4-acetyl-phenyl | | 3-methyl-3H-imidazol-4-ylmethyl | 2-(3-fluoro-benzyl) | 0, N/A |
| 36 | 4-acetyl-phenyl | | 3,5-difluoro-benzyl | 2-(3-fluoro-benzyl) | 0, N/A |
| 37 | 4-acetyl-phenyl | | 3,4,5-trifluoro-benzyl | 2-(3-fluoro-benzyl) | 0, N/A |
| 38 | 4-acetyl-phenyl | | Pentafluoro-phenylmethyl | 2-(3-fluoro-benzyl) | 0, N/A |
| 39 | 4-acetyl-phenyl | | 4-fluoro-benzyl | 2-(3-fluoro-benzyl) | 0, N/A |
| 40 | 4-acetyl-phenyl | | 3,4-difluorobenzyl | Benzyl | 0, N/A |
| 41 | 4-acetyl-phenyl | | 3-chloro-4-fluoro-phenyl | 2-(3-fluoro-benzyl) | 0, N/A |
| 42 | 4-acetyl-phenyl | | 3-chloro-4-fluoro-phenyl | Benzyl | 0, N/A |
| 43 | 4-acetyl-phenyl | | 3,4-difluoro-benzyl | 2-(3-fluoro-benzyl) | 0, N/A |
| 44 | 4-acetyl-phenyl | | 3,4-difluoro-benzyl | Benzyl | 0, N/A |
| 45 | 4-acetyl-phenyl | | Phenyl | Benzyl | 0, N/A |
| 46 | 4-acetyl-phenyl | | Cyclohexylmethyl | Benzyl | 0, N/A |
| 47 | 4-carbamoyl-phenyl | | Cyclohexylmethyl | Benzyl | 0, N/A |
| 48 | 4-acetyl-phenyl | | cyclohexyl | 2-(chloro-benzyl) | 0, N/A |
| 49 | 4-acetyl phenyl | | cyclohexyl | 4-(fluoro-benzyl) | 0, N/A |
| 50 | 4-acetyl phenyl | | cyclohexyl | 2-pyridin-4-yl-ethyl | 0, N/A |
| 51 | 4-acetyl phenyl | | cyclohexyl | 3-(chloro-benzyl) | 0, N/A |
| 52 | 4-acetyl phenyl | | cyclohexyl | 2-thiophen-2-yl | 0, N/A |
| | | | | methyl | |
| 54 | 4-acetyl phenyl | | cyclohexyl | 2-pyridin-3-yl | 0, N/A |
| | | | | methyl | |
| 55 | | 4-acetyl-2-methyl-phenyl | Cyclohexylmethyl | benzyl | 0, N/A |
| 56 | 4-(1hydroxy-ethyl)phenyl | | Cyclohexylmethyl | Benzyl | 0, N/A |
| 57 | | 4-acetyl-3-methyl-phenyl | cyclohexyl | Benzyl | 0, N/A |
| 58 | | 4-acetyl phenyl | cyclohexyl | Benzyl | 0, N/A |
| 59 | | 3-oxo-2,3,dihydro-benzofuran-6-yl | cyclohexyl | Benzyl | 0, N/A |
| 60 | 4-acetyl phenyl | | 3,4,difluoro-phenyl | 2-(3-fluro-benzyl) | 0, N/A |
| 61 | 4-acetyl phenyl | | 3-fluro-phenyl | 2-(3-fluro-benzyl) | 0, N/A |
| 62 | 4-acetyl phenyl | | 2,4,-difluoro-phenyl | 2-(3-fluro-benzyl) | 0, N/A |
| 63 | 4-acetyl phenyl | | 2,3,-difluoro-phenyl | 2-(3-fluro-benzyl) | 0, N/A |

Compounds of the present invention are enumerated below with example numbers and compound numbers. Compound numbers are underlined. The example number will not necessarily equal the compound number but are nevertheless unambiguously correlated.

### Example 1

### Synthesis of (6S,9aS)-6-Cyclohexyl-4,7-dioxo-8-phenethyl-hexahydro-pyrazino[1,2-a]pyrimidine-1-carboxylic acid (4-acetyl-phenyl)-amide 1

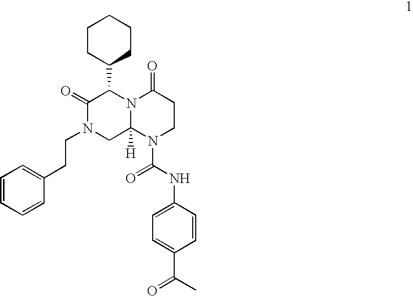

To a 500 mL round bottom flask was added 10.0 g of PEG-grafted Polystyrene hydroxyl resin (0.48 mmol/g), 3.74 g (14.9 mmol) of PPTS (pyridinium p-toluenesulfonate) and 200 mL of dry DCE (1,2-dichloroethane) under Argon. The round bottom flask was fitted with a short-path distillation column and 50 mL of DCE was distilled off at one atmosphere. To the reaction mixture was then added 9.0 mL of bromoacetaldehyde diethyl acetal (11.8 g, 59.8 mmol) in 50 mL of DCE. The short path distillation column was employed again to remove 50 mL of DCE. The addition of the acetal and distillation procedure was repeated. Upon cooling, the resin was filtered, washed three times with DMF (N,N-dimethylformamide) and Dioxane.

To the resulting resin was added a 0.2M solution of Phenethylamine in DMSO (Dimethylsulfoxide). The resulting mixture was shaken at 60° C. over night. Upon return to room temperature, the resin was drained, washed five times with DMF and DCM (Dichloromethane).

To the phenethylamine resin was added a 0.2M solution of DIC (Diisopropylcarbodiimide), HOAT (1-Hydroxy-7-azabenzotriazole), and FMOC-CHG-OH (N-α-Fluorenylmethoxycarbonyl-β-cyclohexyl-L-glycine). The resulting mixture was shaken at room temperature over night. The resin was drained and washed five times with DMF and DCM.

To the Fmoc-cyclohexylglycine resin was added a 20% piperidine solution in DMF. The mixture was shaken for 30 minutes at room temperature then drained, and washed five times with DMF and DCM. To the deprotected amine resin was added a 0.2M solution of DIC (Diisopropylcarbodiimide), HOBT (N-Hydroxybenzotriazole), and FMOC-β-Ala-OH (N-α-Fluorenylmethoxycarbonyl-β-alanine). The mixture was shaken overnight at room temperature. The resulting resin mixture was drained and washed five times with DMF and DCM.

To the Fmoc-β-alanine resin was added a 20% piperidine solution in DMF. The mixture was shaken for a half-hour at room temperature then drained, and washed five times with DMF and DCM. To the β-alanine resin was added a 0.2M solution of 4-Acetylphenyl isocyanate in DCE. The mixture was shaken over night at room temperature. The resulting resin mixture was drained, then washed several times with DMF and DCM.

Addition of formic acid to the resulting resin followed by shaking at room temperature over night afforded (6S,9aS)-6-Cyclohexyl-4,7-dioxo-8-phenethyl-hexahydro-pyrazino[1,2-a]pyrimidine-1-carboxylic acid (4-acetyl-phenyl)-amide in a 24% yield, as a white solid.

¹H NMR: (500 MHz, CDCl₃) δ 1.16 (m, 4H), 1.26 (m, 1H), 1.62 (m, 2H), 1.73 (m, 2H), 1.99 (m, 2H), 2.48 (m, 1H), 2.68 (m, 1H), 2.85 (m, 1H), 2.94 (m, 1H), 3.14 (t, 1H, J=10.99 Hz), 3.29 (dd, 1H, J=4.28, 11.29 Hz), 3.41 (t, 1H, J=11.60 Hz), 3.55 (m, 1H), 3.62 (m, 1H), 3.86 (m, 1H), 5.05 (d, 1H, J=7.02 Hz), 5.95 (dd, 1H, J=4.28, 10.69 Hz), 6.97 (s, 1H), 7.21 (m, 3H), 7.30 (m, 2H), 7.49 (d, 2H, J=8.85 Hz), 7.95 (d, 2H, J=8.85 Hz).

LC/MS: (Method A) m/z 517.30, Rf 1.750, 100% purity.

### Example 2

### (6S,9aS)-6-Cyclohexyl-4,7-dioxo-8-phenethyl-hexahydro-pyrazino[1,2a]pyrimidine-1-carboxylic acid (4-methanesulfonyl-phenyl)-amide 17

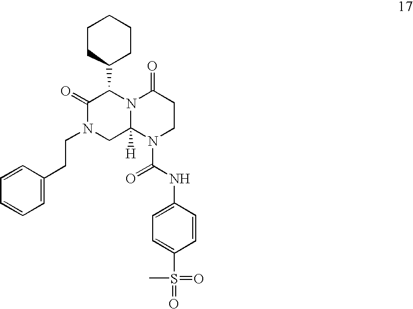

¹HNMR: (300 MHz, CDCl₃) δ 1.16 (m, 5H), 1.61 (m, 4H), 2.00 (m, 2H), 2.50 (m, 1H), 2.68 (m, 1H), 2.92 (m, 2H), 3.04 (s, 3H), 3.16 (m, 1H), 3.34 (m, 2H), 3.57 (m, 2H), 3.92 (m, 1H), 5.03 (d, 1H, J=6.59 Hz), 5.95 (m, 1H), 7.25 (m, 5H), 7.57 (d, 2H, J=8.78 Hz), 7.80(d, 2H, J=8.78 Hz).

LC/MS: (Method A) m/z 553.32, Rf 1.477, 85.1% purity.

### Example 3

### (6S,9aS)-6-Cyclohexyl-4,7-dioxo-8-phenethyl-hexahydro-pyrazino[1,2a]pyrimidine-1-carboxylic acid (4-methanesulfinyl-phenyl)-amide 18

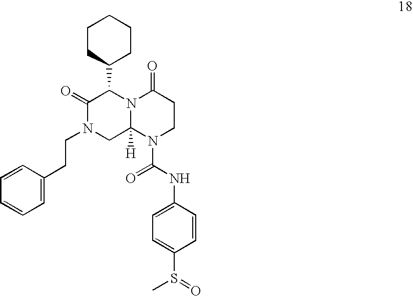

¹HNMR: (300 MHz, CDCl₃) δ 1.16 (m, 5H), 1.61 (m, 4H), 2.00 (m, 2H), 2.50 (m, 1H), 2.68 (m, 1H), 2.92 (m, 2H), 3.04 (s, 3H), 3.16 (m, 1H), 3.34 (m, 2H), 3.57 (m, 2H), 3.92 (m, 1H), 5.03 (d, 1H, J=6.59 Hz), 5.95 (m, 1H), 7.25 (m, 5H), 7.57 (d, 2H, J=8.78 Hz), 7.80(d, 2H, J=8.78 Hz).

LC/MS: (Method A) m/z 537.28, Rf 1.560, 90.0% purity.

### Example 4

### (6S,9aS)-6-Cyclohexyl-4,7-dioxo-8-phenethyl-hexahydro-pyrazino[1,2-a]pyrimidine-1-carboxylic acid (4-methylsulfanyl-phenyl)-amide 19

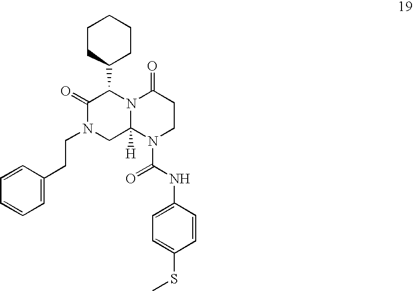

¹HNMR: (500 MHz, CDCl₃) 1.16 (m, 5H), 1.61 (m, 2H), 1.72 (m, 2H), 1.99 (m, 2H), 2.46 (m, 1H), 2.70 (m, 1H), 2.73 (s, 3H), 2.85 (m, 1H), 2.94 (m, 1H),), 3.17 (m, 1H), 3.31 (m, 2H), 3.52 (m, 1H), 3.63 (m, 1H), 4.00 (m, 1H), 5.05 (d, 1H, J=7.02 Hz), 5.96 (m, 1H), 7.26 (m, 5H), 7.53 (m, 4H).

LC/MS: (Method A) m/z 521.23, Rf 1.893, 95.0% purity.

### Example 5

### (6S,9aS)-8-[2-(3-Fluoro-phenyl)-ethyl]-6-isopropyl-4,7-dioxo-hexahydro-pyrazino[1,2-a]pyrimidine-1-carboxylic acid (4-acetyl-phenyl)-amide 20

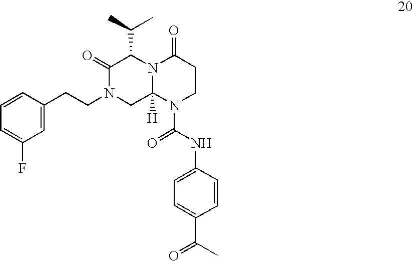

¹HNMR: (300 MHz, CDCl₃) δ 0.98 (d, 3H, J=6.96 Hz), 1.11 (d, 3H, J=6.96 Hz), 2.35 (m, 1H), 2.47 (m, 1H), 2.57 (s, 3H), 2.66 (m, 1H), 2.89 (m, 3H),), 3.30 (m, 2H), 3.46 (m, 1H), 3.55 (m, 1H), 3.98 (m, 1H), 5.02 (d, 1H, J=7.32 Hz), 6.05 (dd, 1H, J=4.75, 9.88 Hz), 6.94 (m, 3H), 7.52 (m, 3H), 7.91 (d, 4H, J=8.42 Hz).

LC/MS: (Method A) m/z 495.36, Rf 1.553, 95.0% purity.

### Example 6

### (6S,9aS)-6-(1S-Methyl-propyl)-8-[2-(3-fluoro-phenyl)-ethyl]-4,7-dioxo-hexahydro-pyrazino[1,2-a]pyrimidine-1-carboxylic acid (4-acetyl-phenyl)-amide 21

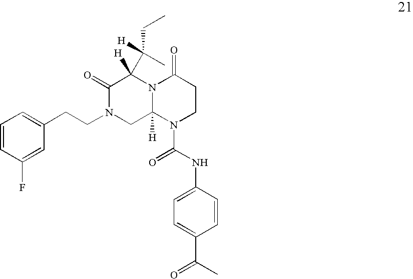

¹HNMR: (300 MHz, CDCl₃) δ 0.89 (t, 3H, J=7.32 Hz), 1.04 (d, 3H, J=6.95 Hz), 1.23 (m, 1H), 1.49 (m, 1H), 2.08 (m, 1H), 2.47 (m, 1H), 2.57 (s, 3H),), 2.68 (m, 1H), 2.88 (m, 3H), 3.34 (m, 2H), 3.47 (m, 1H), 3.56 (m, 1H), 3.96 (m, 1H), 5.10 (d, 1H, J=6.59 Hz), 6.05 (dd, 1H, J=4.39, 10.24 Hz), 6.94 (m, 3H), 7.45 (s, 1H), 7.51 (d, 2H, J=8.78 Hz), 7.92 (d, 2H, J=8.78 Hz).

LC/MS: (Method A) m/z 509.26, Rf 1.623, 98.0% purity.

### Example 7

### (6S,9aS)-6-Benzyl-8-[2-(3-fluoro-phenyl)-ethyl]-4,7-dioxo-hexahydro-pyrazino[1,2-a]pyrimidine-1-carboxylic acid (4-acetyl-phenyl)-amide 22

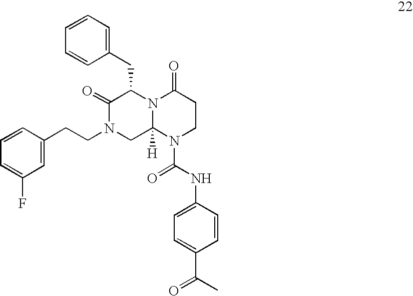

¹HNMR: (300 MHz, CDCl₃) δ 2.40 (m, 1H), 2.50 (m, 1H), 2.59 (s, 3H), 2.89 (m, 2H), 3.02 (m, 1H), 3.21 (m, 1H), 3.30 (m, 1H), 3.40 (m, 2H), 3.56 (m, 3H), 4.04 (m, 1H), 4.96 (dd, 1H, J=3.66, 10.62 Hz), 5.37 (t, 1H, J=5.49 Hz), 6.90 (m, 3H), 7.21 (m, 6H), 7.39 (d, 2H, J=8.79 Hz), 7.94 (d, 2H, J=8.79 Hz).

LC/MS: (Method A) m/z 543.32, Rf 1.607, 97.1% purity.

### Example 8

### (6S,9aS)-8-[2-(3-Fluoro-phenyl)-ethyl]-6-isobutyl-4,7-dioxo-hexahydro-pyrazino[1,2-a]pyrimidine-1-carboxylic acid (4-acetyl-phenyl)-amide 23

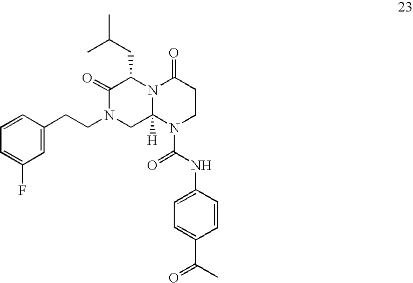

¹HNMR: (300 MHz, CDCl₃) δ 0.95 (dd, 6H, 6.59, 14.64), 1.72 (m, 3H), 2.50 (m, 1H), 2.57 (s, 3H), 2.64 (m, 1H), 2.87 (m, 2H), 3.23 (m, 1H), 3.34 (m, 1H), 3.48 (m, 2H), 3.60 (m, 1H), 3.96 (m, 1H), 5.28 (dd, 1H, J=4.39, 9.88 Hz), 5.90 (dd, 1H, J=4.39, 10.62 Hz), 6.93 (m, 3H), 7.44 (s, 1H), 7.50 (d, 2H, J=8.78 Hz), 7.92 (d, 2H, J=8.78 Hz).

LC/MS: (Method A) m/z 509.30, Rf 1.623, 100% purity.

### Example 9

### (6S,9aS)-8-[2-(3-Fluoro-phenyl)-ethyl]-6-(2-methylsulfanyl-ethyl)-4,7-dioxo-hexahydro-pyrazino[1,2-a]pyrimidine-1-carboxylic acid (4-acetyl-phenyl)-amide 24

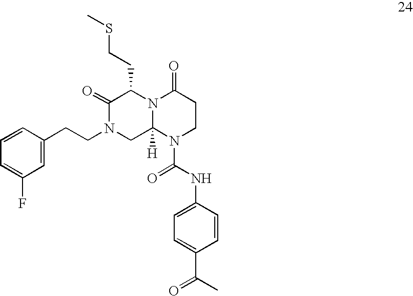

¹HNMR: (300 MHz, CDCl₃) δ 2.08 (s, 3H), 2.35 (m, 1H), 2.50 (m, 1H), 2.50 (m, 2H), 2.57 (s, 3H), 2.59 (m, 3H), 2.89 (m, 3H), 3.32 (d, 1H, J=7.32 Hz), 3.54 (m, 3H), 3.98 (m, 1H), 5.29 (dd, 1H, J=4.76, 9.15 Hz), 5.91 (t, 1H, J=7.32 Hz), 6.93 (m, 3H), 7.44 (s, 1H), 7.49 (d, 2H, J=8.78 Hz), 7.92 (d, 2H, J=8.78 Hz).

LC/MS: (Method A) m/z 527.25, Rf 1.520, 85.0% purity.

### Example 10

### (6S,9aS)-8-[2-(3-Fluoro-phenyl)-ethyl]-6-methyl-4,7-dioxo-hexahydro-pyrazino[1,2-a]pyrimidine-1-carboxylic acid (4-acetyl-phenyl)-amide 25

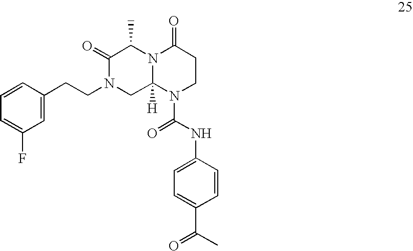

¹HNMR: (300 MHz, CDCl₃) 1.47 (d, 3H, J=6.95 Hz), 2.46 (m, 1H), 2.56 (s, 3H), 2.61 (m, 1H), 2.89 (m, 3H), 3.33 (m, 2H), 3.36 (m, 2H), 4.02 (m, 1H), 5.14 (q, 1H, J=6.96 Hz), 5.90 (m, 1H), 6.91 (m, 3H), 7.51 (d, 2H, J=8.78 Hz), 7.67 (s, 1H) 7.90 (d, 2H, J=8.78 Hz).

LC/MS: (Method A) m/z 467.27, Rf 1.420, 100.0% purity.

### Example 11

### (6S,9aS)-6-Butyl-8-[2-(3-fluoro-phenyl)-ethyl]-4,7-dioxo-hexahydro-pyrazino[1,2-a]pyrimidine-1-carboxylic acid (4-acetyl-phenyl)-amide 26

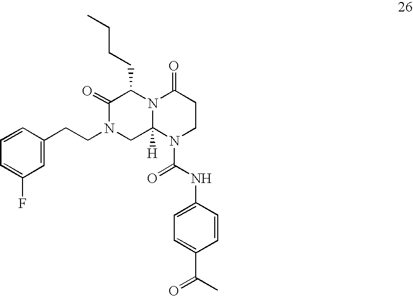

¹HNMR: (300 MHz, CDCl₃) δ 0.86 (t, 3H, J=6.59 Hz), 1.31 (m, 4H), 1.77 (m, 1H), 2.00 (m, 1H), 2.47 (m, 1H), 2.57 (s, 3H), 2.65 (m, 1H), 2.88 (m, 2H), 3.22 (m, 2H), 3.49 (m, 2H), 3.58 (m, 1H), 3.98 (m, 1H), 5.19 (m, 1H), 5.91 (dd, 1H, J=4.76, 10.25 Hz), 6.91 (m, 3H), 7.50 (d, 2H, J=8.78 Hz), 7.57 (s, 1H) 7.92 (d, 2H, J=8.78 Hz).

LC/MS: (Method A) m/z 509.28, Rf 1.633, 100.0% purity.

### Example 12

### (6S,9aS)-8-[2-(3-Fluoro-phenyl)-ethyl]-6-(4-nitro-benzyl)-4,7-dioxo-hexahydro-pyrazino[1,2-a]pyrimidine-1-carboxylic acid (4-acetyl-phenyl)-amide 27

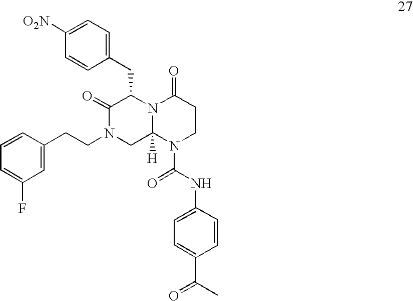

¹HNMR: (300 MHz, CDCl₃) δ 2.40 (m, 2H), 2.58 (s, 3H), 2.91 (m, 3H), 3.35 (m, 5H), 3.56 (m, 1H), 3.97 (m, 1H), 5.41 (m, 1H), 5.69 (m, 1H), 6.92 (m, 3H), 7.37 (d, 2H, J=8.78 Hz), 7.45 (d, 2H, J=8.42 Hz), 7.52 (s, 1H), 7.90 (d, 2H, J=8.42 Hz), 8.05 (d, 2H, J=8.78 Hz).

LC/MS: (Method A) m/z 588.29, Rf 1.617, 98.0% purity.

### Example 13

### (6S,9aS)-6-Cyclohexylmethyl-8-[2-(3-fluoro-phenyl)-ethyl]-4,7-dioxo-hexahydro-pyrazino[1,2-a]pyrimidine-1-carboxylic acid (4-acetyl-phenyl)-amide 28

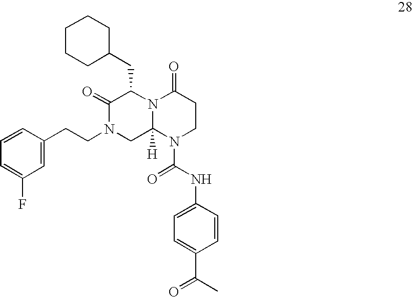

¹HNMR: (300 MHz, CDCl₃) δ 0.92 (m, 2H), 1.21 (m, 4H), 1.65 (m, 5H), 1.89 (m, 2H), 2.47 (m, 1H), 2.57 (s, 3H), 2.66 (m, 1H), 2.87 (m, 3H), 3.23 (m, 1H), 3.41 (m, 3H), 3.97 (m, 1H), 5.30 (dd, 1H, J=4.39, 9.88 Hz), 5.90 (dd, 1H, J=4.39, 10.24 Hz), 6.93 (m, 3H), 7.51 (m, 3H), 7.97 (d, 2H, J=8.78 Hz).

LC/MS: (Method A) m/z 549.34, Rf 1.780, 90.0% purity.

### Example 14

### {1-(4-Acetyl-phenylcarbamoyl)-(6S,9aS)-8-[2-(3-fluoro-phenyl)-ethyl]-4,7-dioxo-octahydro-pyrazino[1,2a]pyrimidin-6-yl}-acetic acid adamantan-2-yl ester 29

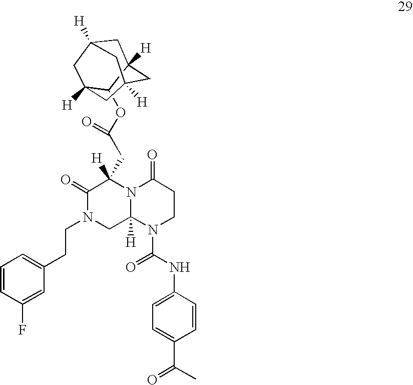

¹HNMR: (300 MHz, CDCl₃) δ 1.71 (br, m, 15H), 2.42 (m, 1H), 2.57 (s, 3H), 2.95 (m, 3H), 3.03 (m, 3H), 3.37 (m, 1H), 4.35 (m, 1H), 4.97 (m, 1H), 5.15 (t, 1H, J=4.75 Hz), 6.36 (dd, 1H, J=2.93, 10.25 Hz), 6.93 (br, 1H), 6.95 (m, 2H), 7.19 (m, 1H), 7.54 (d, 2H, J=8.78 Hz), 7.69 (s, 1H), 7.92 (d, 2H, J=8.78 Hz).

LC/MS: (Method A) m/z 645.42, Rf 1.863, 98.0% purity.

### Example 15

### {1-(4-Acetyl-phenylcarbamoyl)-(6S,9aS)-8-[2-(3-fluoro-phenyl)-ethyl]-4,7-dioxo-octahydro-pyrazino [1,2-a]pyrimidin-6-yl}-acetic acid benzyl ester 30

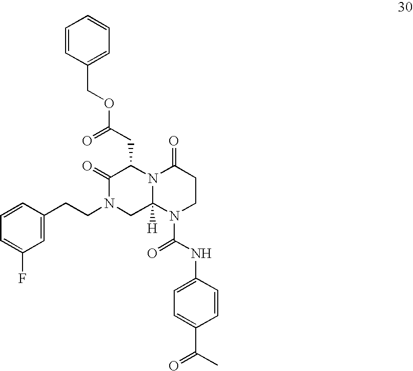

¹HNMR: (300 MHz, CDCl₃) δ 2.40 (m, 2H), 2.58 (s, 3H), 2.91 (m, 3H), 3.35 (m, 5H), 3.56 (m, 1H), 3.97 (m, 1H), 5.41 (m, 1H), 5.69 (m, 1H), 6.92 (m, 3H), 7.37 (d, 2H, J=8.78 Hz), 7.45 (d, 2H, J=8.42 Hz), 7.31 (m, 4H), 7.53 (m, 3H), 7.93 (d, 2H, J=8.78 Hz).

LC/MS: (Method A) m/z 601.29, Rf 1.647, 93.0% purity.

### Example 16

### (4-{1-(4-Acetyl-phenylcarbamoyl)-(6S,9aS)-8-[2-(3-fluoro-phenyl)-ethyl]-4,7-dioxo-octahydro-pyrazino[1,2-a]pyrimidin-6-yl}-butyl)-carbamic acid benzyl ester 31

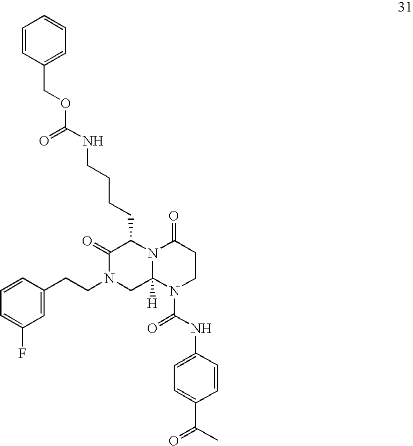

¹HNMR: (300 MHz, CDCl₃). δ 1.47 (m, 4H), 1.96 (m, 2H), 2.41 (m, 2H), 2.56 (s, 3H), 2.86 (m, 2H), 3.15 (m, 2H), 3.30 (m, 3H), 3.51 (m, 2H), 4.00 (m, 1H), 5.10 (br, m, 4H), 5.75 (m, 1H), 6.78 (m, 1H), 6.90 (m, 2H), 7.24 (m, 6H), 7.47 (d, 2H, J=8.42 Hz), 7.89 (d, 2H, J=8.42 Hz).

LC/MS: (Method A) m/z 658.30, Rf 1.670, 98.0% purity.

### Example 17

### (6S,9aS)-6-Benzyloxymethyl-8-[2-(3-fluoro-phenyl)-ethyl]-4,7-dioxo-hexahydro-pyrazino[1,2-a]pyrimidine-1-carboxylic acid (4-acetyl-phenyl)-amide 32

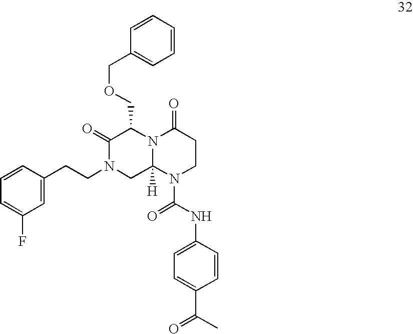

¹HNMR: (300 MHz, CDCl₃) δ2.42 (m, 1H), 2.58 (m, 4H), 2.91 (m, 2H), 3.14 (br, m, 2H), 3.48 (br, m, 3H), 3.86 (dd, 1H, J=2.56, 9.88 Hz), 4.17 (dd, 1H, J=5.12, 13.90 Hz), 4.29 (dd, 1H, J=2.56, 9.52 Hz), 4.52 (m, 2H), 5.16 (br, 1H), 6.25 (dd, 1H, J=3.66, 10.61 Hz), 6.94 (m, 4H), 7.09 (m, 1H), 7.27 (m, 6H), 7.90 (d, 2H, J=8.78 Hz).

LC/MS: m/z (Method A) 573.35, Rf 1.653, 96.0% purity.

### Example 18

### (6S,9aS)-6-(4-Benzyloxy-benzyl)-8-[2-(3-fluoro-phenyl)-ethyl]-4,7-dioxo-hexahydro-pyrazino[1,2-a]pyrimidine-1-carboxylic acid (4-acetyl-phenyl)-amide 33

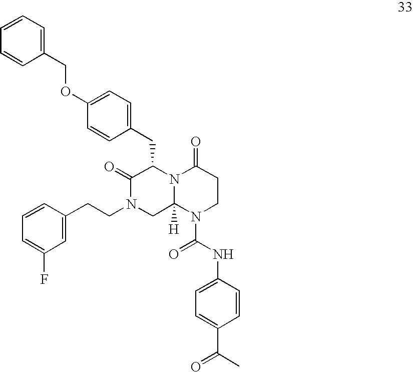

¹HNMR: (300 MHz, CDCl₃) δ 2.40 (m, 1H), 2.47 (s, 3H), 2.53 (m, 1H), 2.87 (m, 2H), 2.98 (m, 1H), 3.20 (m, 3H), 3.34 (m, 2H), 3.53 (m, 1H), 4.06 (m, 1H), 4.87 (m, 2H), 4.98 (dd, 1H, J=3.29, 10.61 Hz), 5.33 (t, 1H, J=5.49 Hz), 6.61 (br, 1H), 6.88 (m, 5H), 7.15 (m, 3H), 7.34 (m, 7H), 7.82 (d, 2H, J=8.78 Hz).

LC/MS: m/z (Method A) 649.24, Rf 1.810, 98.0% purity.

### Example 19

### (6S,9aS)-6-(3,5-Dibromo-4-hydroxy-benzyl)-8-[2-(3-fluoro-phenyl)-ethyl]-4,7-dioxo-hexahydro-pyrazino[1,2-a]pyrimidine-1-carboxylic acid (4-acetyl-phenyl)-amide 34

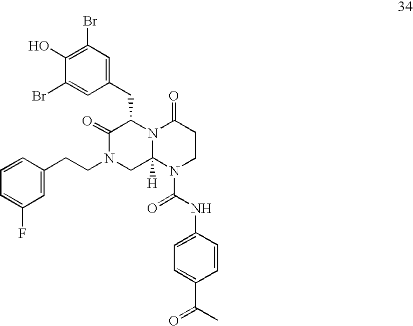

¹HNMR: (300 MHz, CDCl₃) δ 2.31 (m, 1H), 2.45 (s, 1H), 2.56 (s, 3H), 2.87 (m, 2H), 3.10 (m, 2H), 3.29 (m, 3H), 3.56 (m, 3H), 3.96 (m, 1H), 5.26 (m, 1H), 5.65 (dd, 1H, J=3.66, 10.24 Hz), 6.90 (m, 3H), 7.30 (s, 2H), 7.41 (s, br, 1H), 7.51 (m, 3H), 7.89 (d, 2H, J=8.42 Hz).

LC/MS: m/z (Method A) 715.10, Rf 1.650, 84.3% purity.

### Example 20

### (6S,9aS)-6-Ethylsulfanylmethyl-8-[2-(3-fluoro-phenyl)-ethyl]-4,7-dioxo-hexahydro-pyrazino[1,2-a]pyrimidine-1-carboxylic acid (4-acetyl-phenyl)-amide 35

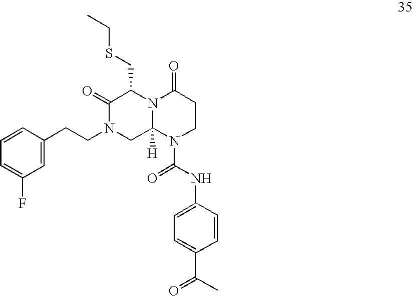

¹HNMR: (300 MHz, CDCl₃). δ 1.23 (t, 3H), 2.47 (m, 1H), 2.57 (s, 3H), 2.60 (m, 3H), 2.91 (m, 2H), 3.29 (m, 5H), 3.61 (m, 2H), 4.10 (dd, 1H, J=4.76, 13.91 Hz), 5.31 (m, 1H), 6.22 (dd, 1H, J=3.66, 10.24 Hz), 6.92 (m, 3H), 7.23 (s, br), 7.41 (s, br, 1H), 7.48 (d, 2H, J=8.79 Hz), 7.89 (d, 2H, J=8.79 Hz).

LC/MS: m/z (Method A) 527.29, Rf 1.543, 90.0% purity.

### Example 21

### (6S,9aS)-8-[2-(3-Fluoro-phenyl)-ethyl]-6-(2-methanesulfonyl-ethyl)-4,7-dioxo-hexahydro-pyrazino[1,2-a]pyrimidine-1-carboxylic acid (4-acetyl-phenyl)-amide 36

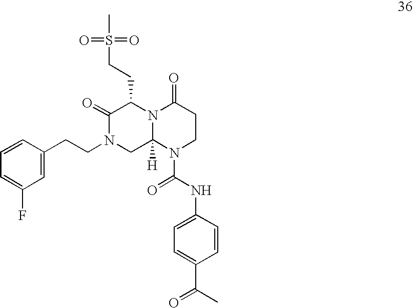

¹HNMR: (300 MHz, CDCl₃) 2.27 (m, 1H), 2.50 (m, 3H), 2.55 (s, 3H), 2.62 (m, 1H), 2.87 (m, 2H), 2.90 (s, 3H), 3.16 (m, 1H), 3.34 (m, 4H), 3.56 (m, 2H), 4.13 (dd, 1H, J=4.75, 13.54 Hz), 5.19 (m, 1H), 5.77 (dd, 1H, J=4.03, 10.24 Hz), 6.87 (m, 3H), 7.22 (br, 1H), 7.49 (d, 2H, J=8.42 Hz), 7.90 (m, 3H).

LC/MS: m/z (Method A) 559.31, Rf 1.330, 95.7% purity.

### Example 22

### (6S,9aS)-8-[2-(3-Fluoro-phenyl)-ethyl]-6-methylsulfanylmethyl-4,7-dioxo-hexahydro-pyrazino[1,2-a]pyrimidine-1-carboxylic acid (4-acetyl-phenyl)-amide 37

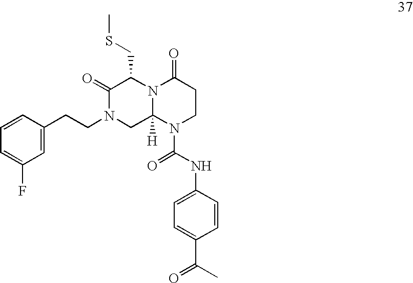

¹HNMR: (300 MHz, CDCl₃) δ 2.15 (s, 3H), 2.49 (m, 1H), 2.57 (s, 3H), 2.67 (m, 1H), 2.91 (m, 2H), 3.17 (m, 2H), 3.35 (m, 2H), 3.56 (m, 3H), 4.10 (m, 1H), 5.33 (m, 1H), 6.20 (dd, 1H, J=4.03, 9.88 Hz), 6.91 (m, 3H), 7.24 (br, s), 7.50 (m, 3H), 7.91 (d, 2H, J=8.78 Hz).

LC/MS: m/z (Method A) 513.34, Rf 1.480, 83.0% purity.

### Example 23

### (6S,9aS)-6-(1R-methyl-propyl)-8-[2-(3-fluoro-phenyl)-ethyl]--4,7-dioxo-hexahydro-pyrazino[1,2-a]pyrimidine-1-carboxylic acid (4-acetyl-phenyl)-amide 39

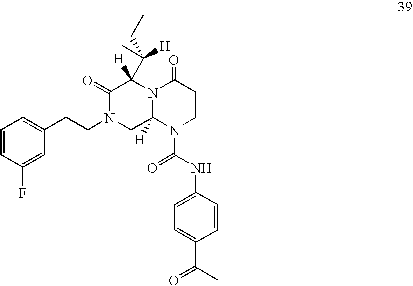

¹HNMR: (300 MHz, CDCl₃) δ 0.95 (m, 6H), 1.27 (m, 1H), 1.68 (m, 1H), 2.16 (m, 1H), 2.47 (m, 1H), 2.57 (s, 3H), 2.69 (m, 1H), 2.88 (m, 2H), 3.31 (m, 2H), 3.48 (m, 1H), 3.57 (m, 2H), 3.97 (m, 1H), 5.14 (d, 1H, J=6.59 Hz), 6.07 (dd, 1H, J=4.76, 10.25 Hz), 6.93 (m, 3H), 7.42 (s, 1H), 7.52 (d, 2H, J=8.79 Hz), 7.92 (d, 2H, J=8.79 Hz).

LC/MS: m/z (Method A) 509.27, Rf 1.630, 98.0% purity.

### Example 24

### (6S,9aS)-6-(2-Carbamoyl-ethyl)-8-[2-(3-fluoro-phenyl)-ethyl]-4,7-dioxo-hexahydro-pyrazino[1,2-a]pyrimidine-1-carboxylic acid (4-acetyl-phenyl)-amide 40

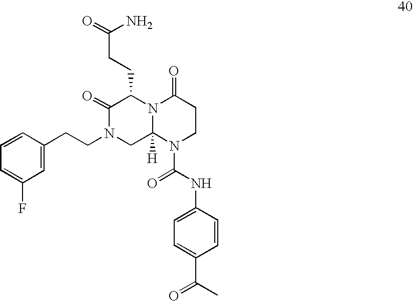

¹HNMR: (300 MHz, CDCl₃) δ 2.42 (m, 6H), 2.55 (m, 1H), 2.57 (s, 3H), 2.80 (m, 1H), 2.91 (m, 2H), 3.07 (m, 1H), 3.34 (m, 2H), 4.29 (m, 1H), 5.26 (m, 1H), 5.55 (dd, 1H, J=4.03, 10.25 Hz), 5.71 (br, s, 1H), 6.07 (br, s, 1H), 6.60 (td, 1H, J=1.83, 8.42 Hz), 6.92 (m, 2H), 7.15 (m, 1H), 7.45 (d, 2H, J=8.78 Hz), 7.92 (d, 2H, J=8.78 Hz).

LC/MS: m/z (Method A) 524.25, Rf 1.330, 98.0% purity.

### Example 25

### (6S,9aS)-6-Benzylsulfanylmethyl-8-[2-(3-fluoro-phenyl)-ethyl]-4,7-dioxo-hexahydro-pyrazino [1,2-a]pyrimidine-1-carboxylic acid (4-acetyl-phenyl)-amide 41

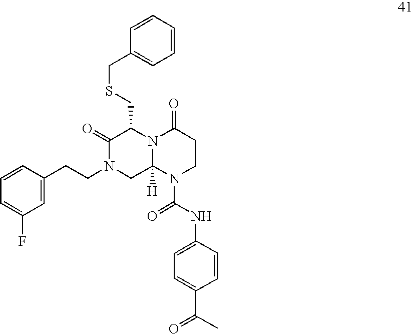

¹HNMR: (300 MHz, CD₃OD) δ 0.87 (m, 1H), 1.02 (s, 3H), 1.08 (m, 1H), 1.39 (m, 2H), 1.58 (m, 1H), 1.76 (m, 2H), 1.92 (m, 2H), 2.09 (m, 2H), 2.17 (s, 2H), 2.60 (m, 1H), 3.65 (m, 1H), 4.70 (dd, 1H, J=3.66, 10.24 Hz), 5.39 (m, 1H), 5.52 (m, 2H), 5.72 (m, 5H), 5.81 (br, s, 1H), 6.06 (d, 2H, J=8.79 Hz), 6.42 (d, 2H, J=8.79 Hz).

LC/MS: m/z (Method A) 589.29, Rf 1.700, 90.0% purity.

### Example 26

### (6S,9aS)-8-[2-(3-Fluoro-phenyl)-ethyl]-6-(2-methanesulfinyl-ethyl)-4,7-dioxo-hexahydro-pyrazino [1,2-a]pyrimidine-1-carboxylic acid (4-acetyl-phenyl)-amide 42

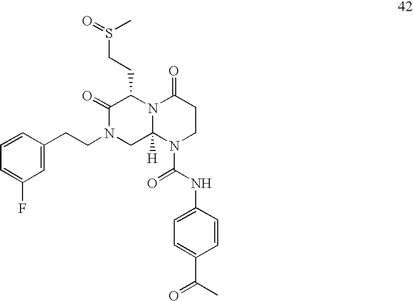

¹HNMR: (300 MHz, CDCl₃, 2:1 Mixture of diastereomers) δ 2.30 (m, 1H), 2.48 (m, 2H), 2.55 & 2.56 (s, 3H), 2.59 (m, 1H), 2.91 (m, 4H), 3.15-3.39 (m, 4H), 3.57 (m, 1H), 4.16-4.37 (m, 1H), 5.21 & 5.45 (m, 1H), 5.69 & 5.78 (dd, 1H, 3.66, 10.25 Hz), 6.58 & 6.90 (m, 3H), 7.09-7.24 (m, 1H), 7.41-7.50 (m, 2H), 7.88-7.92 (m, 2H).

LC/MS: m/z (Method A) 543.18, Rf 1.300, 95.0% purity.

### Example 27

### (6S,9aS)-8-[2-(3-Fluoro-phenyl)-ethyl]-4,7-dioxo-6-piperidin-4-yl-hexahydro-pyrazino[1,2-a]pyrimidine-1-carboxylic acid (4-acetyl-phenyl)-amide 43

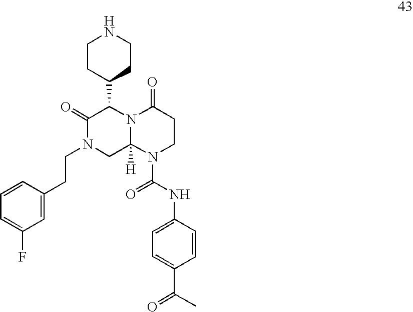

¹HNMR: (500 MHz, CD₃OD) 1.31 (m, 4H), 1.44 (m, 1H), 1.55 (m, 2H), 1.72 (m, 1H), 2.08 (m, 1H), 2.22 (m, 1H), 2.47 (m, 1H), 2.60 (s, 3H), 2.70 (m, 1H), 2.86 (m, 1H), 2.97 (m, 3H), 3.48 (m, 2H), 3.89(m, 1H), 4.19 (dd, 1H, J=6.10, 14.95 Hz), 4.95 (d, 1H, J=8.24 Hz), 6.03 (dd, 1H, J=4.27, 10.68 Hz), 6.99 (td, 1H, J=2.44, 8.55 Hz), 7.10 (m, 2H), 7.34 (m, 1H), 7.64 (d, 2H, J=8.55 Hz), 7.99 (d, 2H, J=8.55 Hz).

LC/MS: m/z (Method A) 536.39, Rf 1.280, 90.0% purity.

### Example 28

### (6S,9aS)-8-[2-(3-Fluoro-phenyl)-ethyl]-4,7-dioxo-6-phenylmethanesulfinylmethyl-hexahydro-pyrazino[1,2-a]pyrimidine-1-carboxylic acid (4-acetyl-phenyl)-amide 44

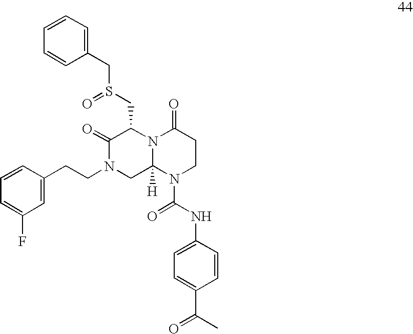

¹HNMR: (500 MHz, CD₃OD, 1:1 Mixture of diastereomers). δ 1.93 (s, 2H), 2.48 (m, 1H), 2.60 (s, 3H), 2.97 (m, 2H), 3.41-3.56 (m, 3H), 3.66 (m, 2H), 3.79 (m, 1H), 4.06 (m, 1H), 4.20 (m, 1H), 4.52 & 4.60 (m, 1H), 5.48-5.55 (m, 1H), 5.99 & 6.06 (dd, 1H, J=3.66, 10.38 Hz), 6.95 (m, 1H), 7.09 (m, 2H), 7.32 (m, 1H), 7.38 (m, 4H), 7.44 (m, 1H), 7.60 (m, 2H), 7.97 (m, 2H).

LC/MS: (Method A) m/z 605.17, Rf 1.553, 99.0% purity.

### Example 29

### (6S,9aS)-8-[2-(3-Fluoro-phenyl)-ethyl]-4,7-dioxo-6-phenylmethanesulfonylmethyl-hexahydro-pyrazino[1,2-a]pyrimidine-1-carboxylic acid (4-acetyl-phenyl)-amide 45

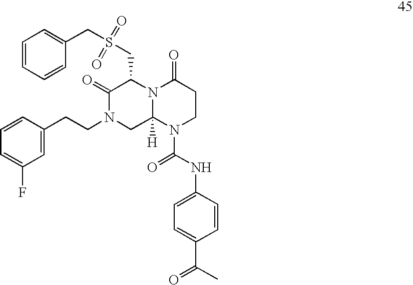

¹HNMR: (500 MHz, CD₃OD). δ 2.46 (m, 1H), 2.59 (s, 3H), 2.67 (m, 1H), 2.95 (m, 2H), 3.48 (m, 2H), 3.65 (m, 3H), 3.78 (m, 2H), 4.17 (m, 1H), 4.52 (m, 2H), 5.55 (m, 1H), 6.20 (dd, 1H, J=3.66, 10.07 Hz), 6.96 (m, 1H), 7.09 (m, 2H), 7.39 (m, 6H), 7.59 (d, 2H, J=8.85 Hz), 7.97 (d, 2H, J=8.85 Hz).

LC/MS: (Method A) m/z 621.17, Rf 1.543, 99.0% purity.

### Example 30

### (6S,9aS)-8-[2-(3-Fluoro-phenyl)-ethyl]-4,7-dioxo-6-phenyl-hexahydro-pyrazino[1,2-a]pyrimidine-1-carboxylic acid (4-acetyl-phenyl)-amide 46

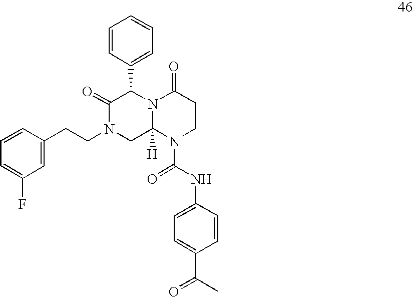

¹HNMR: (500 MHz, CD₃OD). δ 2.47 (m, 1H), 2.53 (s, 3H), 2.69 (m, 1H), 3.07 (m, 2H), 3.31 (m, 1H), 3.54 (m, 2H), 3.69 (m, 1H), 3.92 (m, 1H), 4.12 (dd, 1H, J=5.80, 14.65 Hz), 5.85 (dd, 1H, J=4.27, 10.37 Hz), 6.29 (s, 1H), 7.09 (m, 2H), 7.29 (m, 7H), 7.48 (d, 2H, J=8.55 Hz), 7.90 (d, 2H, J=8.55 Hz).

LC/MS: (method B) m/z 529.29, Rf 1.813, 100% purity.

### Example 31

### (6S,9aS)-6-(1R-Methyl-propyl)-8-[2-(3-fluoro-phenyl)-ethyl]-(S,S)-4,7-dioxo-hexahydro-pyrazino[1,2-a]pyrimidine-1-carboxylic acid (4-acetyl-phenyl)-amide 47

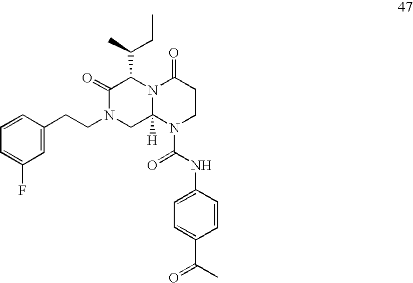

¹HNMR: (500 MHz, CDCl₃). δ 0.89 (t, 3H, J=7.33 Hz), 1.03 (d, 3H, J=6.71 Hz), 1.23 (m, 1H), 1.49 (m, 1H), 2.08 (m, 1H), 2.49 (m, 1H), 2.57 (s, 3H), 2.69 (m, 1H), 2.95 (m, 2H), 3.32 (m, 1H), 3.44 (m, 2H), 3.61 (m, 2H), 3.97 (m, 1H), 5.08 (d, 1H, J=6.71 Hz), 6.05 (dd, 1H, J=4.27, 10.38 Hz), 7.01 (m, 1H), 7.08 (m, 1H), 7.22 (m, 2H), 7.27 (s, br, 1H), 7.51 (d, 2H, J=8.85 Hz), 7.93 (d, 2H, J=8.85 Hz).

LC/MS: (method B) m/z 509.31, Rf 3.443, 100% purity.

### Example 32

### (6S,9aS)-6-Cyclohexyl-8-[2-(3-fluoro-phenyl)-ethyl]-4,7-dioxo-hexahydro-pyrazino[1,2-a]pyrimidine-1-carboxylic acid (4-acetyl-phenyl)-amide 48

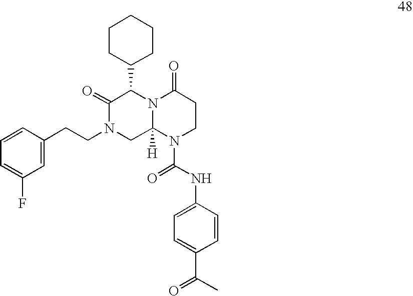

¹HNMR: (500 MHz, CDCl₃). δ 1.13 (m, 5H), 1.59 (m, 2H), 1.69 (m, 2H), 1.93 (m, 2H), 2.46 (m, 1H), 2.57 (s, 3H), 2.68 (m, 1H), 2.93 (m, 2H), 3.32 (m, 1H), 3.42 (m, 2H), 3.53 (m, 1H), 3.65 (m, 1H), 4.00 (m, 1H), 5.00 (d, 1H, J=7.32 Hz), 6.01 (dd, 1H, J=4.27, 10.38 Hz), 7.01 (m, 1H), 7.07 (m, 1H), 7.21 (m, 2H), 7.53 (m, 3H), 7.92 (d, 2H, J=8.54 Hz).

LC/MS: (method B) m/z 535.35, Rf 3.570, 100% purity.

### Example 33

### (6S,9aS)-8-[2-(3-Fluoro-phenyl)-ethyl]-4,7-dioxo-6-(tetrahydro-pyran-4-yl)-hexahydro-pyrazino[1,2-a]pyrimidine-1-carboxylic acid (4-acetyl-phenyl)-amide 52

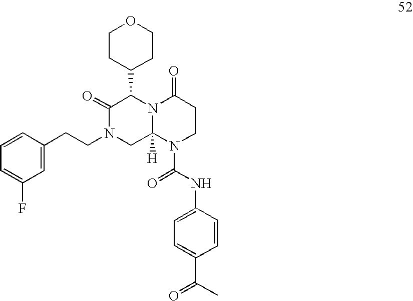

¹HNMR: (500 MHz, CDCl₃). δ 1.49 (m, 4H), 1.80 (m, 1H), 2.22 (m, 1H), 2.47 (m, 1H), 2.57 (s, 3H), 2.67 (m, 1H), 2.88 (m, 2H), 3.35 (m, 4H), 3.52 (m, 1H), 3.70 (m, 1H), 3.94 (m, 3H), 5.05 (d, 1H, J=7.32 Hz), 5.98 (dd, 1H, J=4.02, 10.25 Hz), 6.93 (m, 3H), 7.51 (d, 2H, J=8.78 Hz), 7.76 (br, s, 1H), 7.91 (d, 2H, J=8.78 Hz), 8.05 (s, 1H).

LC/MS: (method A) m/z 537.44, Rf 1.853, 93.2% purity.

### Example 34

### (4S,12S)-2-[2-(3-Fluoro-phenyl)-ethyl]-3,5-dioxo-4-(tetrahydro-pyran-4-yl)-1,3,4,12a-tetrahydro-2H,5H-2,4a,12-triaza-naphthacene-12-carboxylic acid (4-acetyl-phenyl)-amide 53

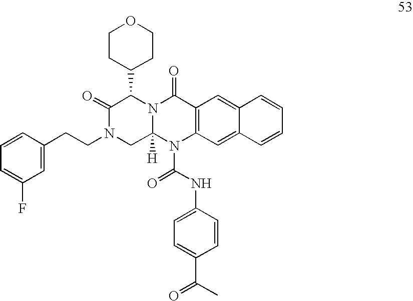

¹HNMR: (300 MHz, CDCl₃) 1.61 (m, 1H), 1.74 (m, 1H), 1.92 (m, 1H), 2.43 (m, 1H), 2.57 (s, 3H), 2.84 (m, 2H), 3.25 (m, 2H), 3.35-3.63 (br, m, 5H), 3.99 (m, 2H), 5.15 (d, 1H, J=6.95 Hz), 6.39 (dd, 1H, J=4.39, 10.25 Hz), 6.66 (td, 1H, J=2.19, 8.41 Hz), 6.85 (m, 2H), 7.12 (m, 1H), 7.48 (d, 1H, J=8.78 Hz), 7.58-7.73 (m, 4H), 7.84 (d, 1H, J=8.05 Hz), 7.94 (d, 2H, J=8.78 Hz), 8.04 (m, 1H), 8.75 (s, br, 1H).

LC/MS: (method A) m/z 635.44, Rf 2.377, 92.2% purity.

### Example 35

### (6S,9aS)-8-[2-(3-Fluoro-phenyl)-ethyl]-6-(3-methyl-3H-imidazol-4-ylmethyl)-4,7-dioxo-hexahydro-pyrazino[1,2-a]pyrimidine-1-carboxylic acid (4-acetyl-phenyl)-amide 54

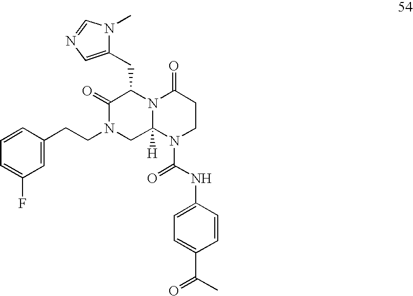

¹HNMR: (300 MHz, CDCl₃). δ 2.27 (m, 1H), 2.32 (m, 2H), 2.57 (s, 3H), 2.81 (m, 3H), 3.04-3.21 (br, m, 1H), 3.22-3.59 (br, m, 4H), 3.61 (s, 3H), 4.40 (dd, 1H, J=5.86, 13.91 Hz), 5.43 (m, 1H), 6.18 (m, 1H), 6.69-6.84 (br, m, 4H), 7.17 (m, 1H), 7.30 (br, s, 1H), 7.54 (d, 2H, J=8.78 Hz), 7.93 (d, 2H, J=8.78 Hz).

LC/MS: (method B) m/z 547.46, Rf 1.957, 92.6% purity.

### Example 36

### (6S,9aS)-6-(3,5-Difluoro-benzyl)-8-[2-(3-fluoro-phenyl)-ethyl]-4,7-dioxo-hexahydro-pyrazino[1,2-a]pyrimidine-1-carboxylic acid (4-acetyl-phenyl)-amide 55

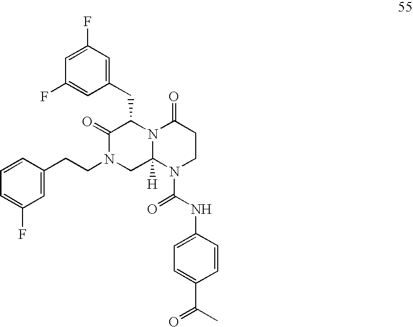

¹HNMR: (300 MHz, CDCl₃). δ 2.42 (m, 1H), 2.53 (m, 1H), 2.59 (s, 3H), 2.92 (m, 2H), 3.16 (m, 1H), 3.26-3.40 (br, m, 4H), 3.61 (m, 2H), 3.91 (m, 1H), 5.36 (t, 1H, J=6.22 Hz), 5.46 (dd, 1H, J=4.02, 10.25 Hz), 6.62 (tt, 1H, J=2.20, 9.15 Hz), 6.77 (m, 2H), 6.90-7.01 (m, 4H), 7.44 (d, 2H, J=8.78 Hz), 7.95 (d, 2H, J=8.78 Hz), 8.04 (br, s, 1H).

LC/MS: (method B) m/z 579.43, Rf 2.810, 90.1% purity.

### Example 37

### (6S,9aS)-8-[2-(3-Fluoro-phenyl)-ethyl]-4,7-dioxo-6-(3,4,5-trifluoro-benzyl)-hexahydro-pyrazino[1,2-a]pyrimidine-1-carboxylic acid (4-acetyl-phenyl)-amide 56

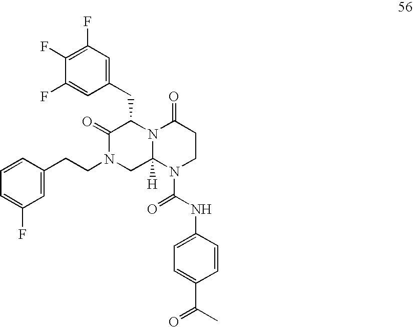

¹HNMR: (300 MHz, CDCl₃). δ 2.39 (m, 1H), 2.50 (m, 1H), 2.58 (s, 3H), 2.88 (m, 3H), 3.21 (m, 3H), 3.35 (m, 2H), 3.57 (m, 2H), 3.71 (m, 2H), 3.94 (m, 1H), 5.32 (m, 1H), 5.64 (dd, 1H, J=3.66, 10.61 Hz), 6.89 (m, 5H), 7.46 (m, 3H), 7.93 (d, 2H, J=8.42 Hz), 8.06 (br, s, 1H).

LC/MS: (method B) m/z 597.42, Rf 2.900, 91.4% purity.

### Example 38

### (6S,9aS)-8-[2-(3-Fluoro-phenyl)-ethyl]-4,7-dioxo-6-pentafluorophenylmethyl-hexahydro-pyrazino [1,2a]pyrimidine-1-carboxylic acid (4-acetyl-phenyl)-amide 57

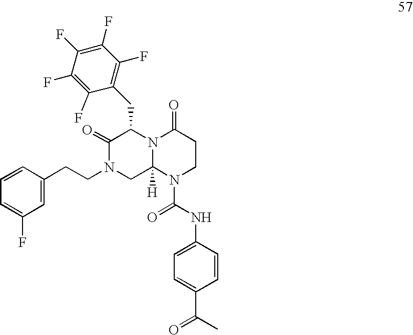

¹HNMR: (300 MHz, CDCl₃). δ 2.37 (m, 1H), 2.50 (m, 1H), 2.58 (s, 3H), 2.88 (m, 2H), 3.16 (m, 1H), 3.38 (m, 2H), 3.52 (m, 1H), 3.68 (m, 2H), 3.85 (m, 1H), 3.97 (m, 1H), 5.41 (dd, 1H, J=5.40, 10.25 Hz), 5.95 (m, 1H), 6.94 (m, 3H), 7.50 (d, 2H, J=8.78 Hz), 7.58 (s, 1H), 7.93 (d, 2H, J=8.78 Hz), 8.08 (br, s, 1H).

LC/MS: (method B) m/z 633.44, Rf 2.883, 91.0% purity.

### Example 39

### (6S,9aS)-6-(4-Fluoro-benzyl)-8-[2-(3-fluoro-phenyl)-ethyl]-4,7-dioxo-hexahydro-pyrazino[1,2-a]pyrimidine-1-carboxylic acid (4-acetyl-phenyl)-amide 58

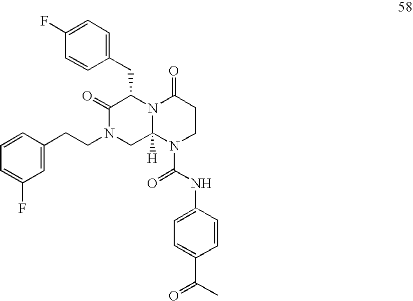

¹HNMR: (300 MHz, CDCl₃). δ 2.40 (m, 1H), 2.48 (m, 1H), 2.59 (s, 3H), 2.90 (m, 2H), 3.14 (m, 1H), 3.28 (m, 4H), 3.59 (m, 2H), 3.96 (m, 1H), 5.37 (m, 2H), 6.91 (m, 6H), 7.16 (m, 2H), 7.43 (d, 2H, J=8.78 Hz), 7.95 (d, 2H, J=8.78 Hz), 8.10 (br, s).

LC/MS: (method B) m/z 561.44, Rf 2.730, 96.1% purity.

### Example 40

### (6S,9aS)-6-(3,4-Difluoro-phenyl)-4,7-dioxo-8-phenethyl-hexahydro-pyrazino[1,2-a]pyrimidine-1-carboxylic acid (4-acetyl-phenyl)-amide 59

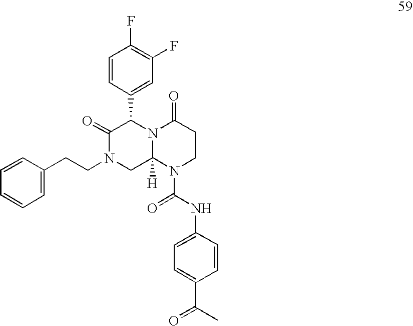

¹HNMR: (300 MHz, CDCl₃). δ 2.47 (m, 1H), 2.55 (s, 3H), 2.63 (m, 1H), 3.00 (m, 2H), 3.27 (m, 1H), 3.46 (m, 2H), 3.69 (m, 2H), 3.98 (m, 1H), 5.76 (m, 1H), 6.32 (s, 1H), 6.94 (m, 1H), 7.05 (m, 1H), 7.25 (m, 6H), 7.43 (d, 2H, J=8.41 Hz), 7.57 (br, s, 1H), 7.86 (d, 2H, J=8.41 Hz).

LC/MS: (method B) m/z 529.29, Rf 1.813, 100% purity.

### Example 41

### (6S,9aS)-6-(4-Chloro-3-fluoro-phenyl)-8-[2-(3-fluoro-phenyl)-ethyl]-4,7-dioxo-hexahydro-pyrazino[1,2-a]pyrimidine-1-carboxylic acid (4-acetyl-phenyl)-amide 60

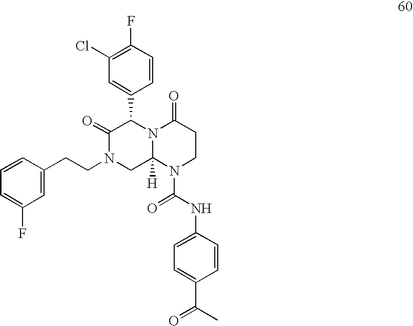

¹HNMR: (300 MHz, CDCl₃). δ 2.48 (m, 1H), 2.54 (s, 3H), 2.66 (m, 1H), 2.98 (m, 2H), 3.44 (m, 4H), 3.71 (m, 1H), 4.00 (m, 1H), 5.80 (dd, 1H, J=4.03, 10.61 Hz), 6.32 (s, 1H), 7.04 (m, 5H), 7.29 (br, s), 7.44 (m, 3H), 7.61 (s, 1H), 7.86 (d, 2H, J=8.78 Hz).

LC/MS: (method A) m/z 582.13, Rf 1.750, 98.0% purity.

### Example 42

### (6S,9aS)-6-(4-Chloro-3-fluoro-phenyl)-4,7-dioxo-8-phenethyl-hexahydro-pyrazino[1,2-a]pyrimidine-1-carboxylic acid (4-acetyl-phenyl)-amide 61

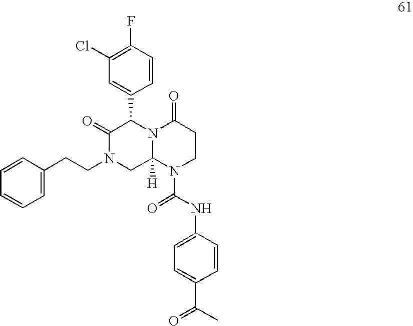

¹HNMR: (300 MHz, CDCl₃). δ 2.50 (m, 1H), 2.55 (s, 3H), 2.68 (m, 1H), 2.94 (m, 2H), 3.26 (m, 1H), 3.41 (m, 2H), 3.70 (m, 2H), 3.95 (m, 1H), 5.75 (dd, 1H, J=4.40, 10.62 Hz), 6.34 (s, 1H), 7.04 (m, 2H), 7.25 (m, 4H), 7.44 (m, 4H), 7.87 (d, 2H, J=8.78 Hz).

LC/MS: (method A) m/z 564.21, Rf 1.733, 88.0% purity.

### Example 43

### (6S,9aS)-6-(3,4-Difluoro-benzyl)-8-[2-(3-fluoro-phenyl)-ethyl]-4,7-dioxo-hexahydro-pyrazino[1,2-a]pyrimidine-1-carboxylic acid (4-acetyl-phenyl)-amide 62

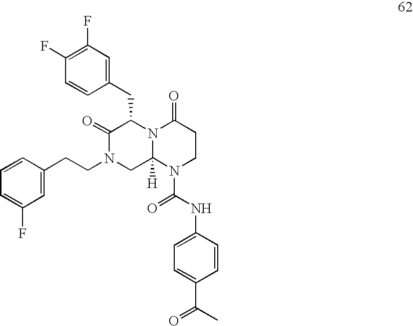

¹HNMR: (300 MHz, CDCl₃). δ 2.36 (m, 1H), 2.47 (m, 1H), 2.58 (s, 3H), 2.87 (m, 2H), 3.20 (m, 3H), 3.33 (m, 2H), 3.58 (m, 2H), 3.96 (m, 1H), 5.34 (m, 1H), 5.56 (dd, 1H, J=4.03, 10.62 Hz), 6.97 (m, 6H), 7.24 (s, br, 1H), 7.38 (br, s, 1H), 7.46 (d, 2H, J=8.42 Hz), 7.92 (d, 2H, J=8.42 Hz).

LC/MS: (method A) m/z 579.16, Rf 1.710, 98.0% purity.

### Example 44

### (6S,9aS)-6-(3,4-Difluoro-benzyl)-4,7-dioxo-8-phenethyl-hexahydro-pyrazino[1,2-a]pyrimidine-1-carboxylic acid (4-acetyl-phenyl)-amide 63

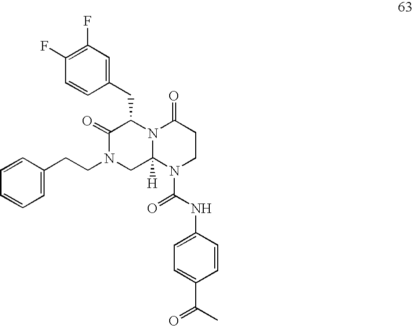

¹HNMR: (300 MHz, CDCl₃). δ 2.35 (m, 1H), 2.47 (m, 1H), 2.58 (s, 3H), 2.90 (m, 2H), 3.10 (m, 1H), 3.23 (m, 3H), 3.56 (m, 3H), 3.92 (m, 1H), 5.35 (m, 1H), 5.47 (dd, 1H, J=3.66, 10.61 Hz), 7.00 (m, 4H), 7.19 (m, 3H), 7.29 (m, 1H), 7.45 (d, 2H, J=8.78 Hz), 7.93 (d, 2H, J=8.78 Hz).

LC/MS: (method A) m/z 561.21, Rf 1.697, 90.0% purity.

### Example 45

### (6S,9aS)-4,7-Dioxo-8-phenethyl-6-phenyl-hexahydro-pyrazino[1,2-a]pyrimidine-1-carboxylic acid (4-acetyl-phenyl)-amide 64

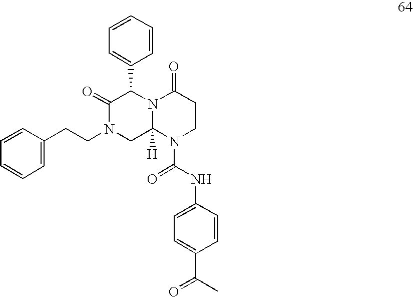

¹HNMR: (300 MHz, CDCl₃) δ 2.45 (m, 1H), 2.54 (s, 3H), 2.62 (m, 1H), 2.98 (br, m, 2H), 3.27 (m, 1H), 3.35 (m, 1H), 3.48 (m, 1H), 3.70 (m, 2H), 3.92 (m, 1H), 5.77 (dd, 1H, J=4.40, 10.24 Hz), 6.42 (s, 1H), 7.28 (m, 10H), 7.33 (br, s, 1H), 7.42 (m, 3H), 7.85 (d, 2H, J=8.78 Hz).

LC/MS: (method A) m/z 511.15, Rf 1.577, 85.0% purity.

### Example 46

### (6S,9aS)-6-Cyclohexylmethyl-4,7-dioxo-8-phenethyl-hexahydro-pyrazino[1,2-a]pyrimidine-1-carboxylic acid (4-acetyl-phenyl)-amide 65

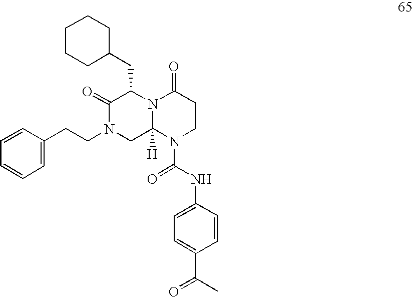

¹H NMR: (500 MHz, CD₃OD) δ 0.96 (m, 2H), 1.22 (m, 4H), 1.70 (m, 6H), 2.02 (m, 1H), 2.42 (m, 1H), 2.59 (s, 3H), 2.67 (m, 1H), 2.93 (m, 2H), 3.35 (m, 1H), 3.47 (m, 2H), 3.61 (m, 2H), 4.14 (dd, 1H, J=5.04, 14.50 Hz), 5.23 (dd, 1H, J=5.04, 9.46 Hz), 5.96 (dd, 1H, J=4.41, 10.72 Hz), 7.27 (m, 3H), 7.32 (m, 2H), 7.60 (d, 2H, J=8.83 Hz), 7.92 (br, s, 1H), 7.98 (d, 2H, J=8.83 Hz).

Chiral HPLC Rf=12.73 minutes.

LC/MS: (method A) m/z 531.25, Rf 2.007, 96.4% purity.

### Example 47

### (6S,9aS)-6-Cyclohexylmethyl-4,7-dioxo-8-phenethyl-hexahydro-pyrazino[1,2-a]pyrimidine-1-carboxylic acid (4-carbamoyl-phenyl)-amide 111

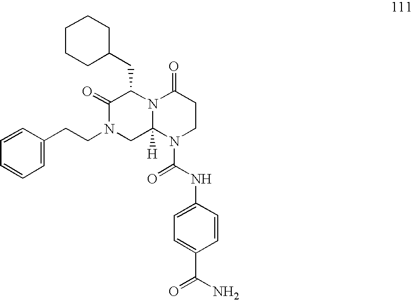

LC/MS: (method A) m/z 532.29, Rf 1.583, 93.3% purity.

### Example 48

### (6S,9aS)-8-[2-(2-Chloro-phenyl)-ethyl]-6-cyclohexyl-4,7-dioxo-hexahydro-pyrazino[1,2-a]pyrimidine-1-carboxylic acid (4-acetyl-phenyl)-amide 116

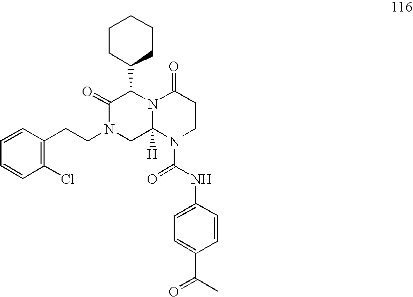

¹H NMR: (300 MHz, CDCl₃) 1.17 (m, 5H), 1.60 (m, 2H), 1.72 (m, 2H), 1.98 (m, 2H), 2.53 (m, 1H), 2.58 (s, 3H), 2.68 (m, 1H), 3.02 (m, 2H), 3.24 (m, 1H), 3.36 (m, 1H), 3.40 (m, 1H), 3.61 (m, 2H), 3.87 (m, 1H), 5.05 (d, 1H, J=7.32 Hz), 6.00 (dd, 1H, J=4.39, 10.61 Hz), 6.88 (br, s, 1H), 7.21 (m, 3H), 7.34 (m, 1H), 7.49 (d, 2H, J=8.78 Hz), 7.95 (d, 2H, J=8.78 Hz).

LC/MS: (method A) m/z 551.41, Rf 1.817, 97.0% purity.

### Example 49

### (6S,9aS)-6-Cyclohexyl-8-[2-(4-fluoro-phenyl)-ethyl]-4,7-dioxo-hexahydro-pyrazino[1,2-a]pyrimidine-1-carboxylic acid (4-acetyl-phenyl)-amide 122

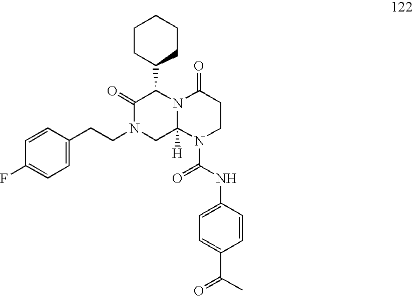

¹H NMR: (300 MHz, CDCl₃) 1.17 (m, 5H), 1.59 (m, 2H), 1.71 (m, 2H), 1.95 (m, 2H), 2.50 (m, 1H), 2.58 (s, 3H), 2.68 (m, 1H), 2.90 (m, 2H), 3.20 (m, 1H), 3.35 (m, 1H), 3.55 (m, 2H), 3.72 (m, 1H), 3.90 (m, 1H), 5.04 (d, 1H, J=6.96 Hz), 5.98 (dd, 1H, J=4.39, 10.61 Hz), 6.99 (m, 2H), 7.16 (m, 2H), 7.19 (br, s, 1H), 7.50 (d, 2H, J=8.78 Hz), 7.94 (d, 2H, J=8.78 Hz).

LC/MS: (method A) m/z 535.45, Rf 1.757, 94.7% purity.

### Example 50

### (6S,9aS)-6-Cyclohexyl-4,7-dioxo-8-(2-pyridin-4-yl-ethyl)-hexahydro-pyrazino[1,2-a]pyrimidine-1-carboxylic acid (4-acetyl-phenyl)-amide 132

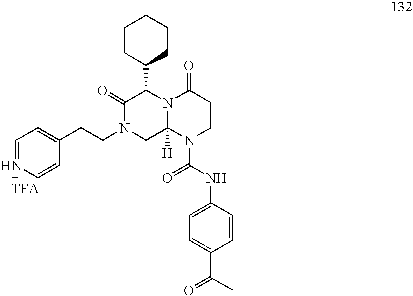

¹HNMR: (300 MHz, CDCl₃) δ 1.18 (m, 5H), 1.59 (m, 2H), 1.73 (m, 2H), 1.96 (m, 2H), 2.53 (m, 1H), 2.58 (s, 3H), 2.70 (m, 1H), 2.95 (m, 2H), 3.17 (m, 1H), 3.39 (m, 2H), 3.63 (m, 2H), 3.89 (m, 1H), 5.06 (d, 1H, J=6.95 Hz), 5.97 (dd, 1H, J=4.02, 10.24 Hz), 7.23 (m, 2H), 7.30 (m, 2H), 7.51 (d, 2H, J=8.42 Hz), 7.94 (d, 2H, J=8.42 Hz), 8.07 (br, s, 1H), 8.55 (br, s, 1H).

LC/MS: (method A) m/z 518.41, Rf 1.313, 94.6% purity.

### Example 51

### (6S,9aS)-8-[2-(3-Chloro-phenyl)-ethyl]-6-cyclohexyl-4,7-dioxo-hexahydro-pyrazino[1,2-a]pyrimidine-1-carboxylic acid (4-acetyl-phenyl)-amide 135

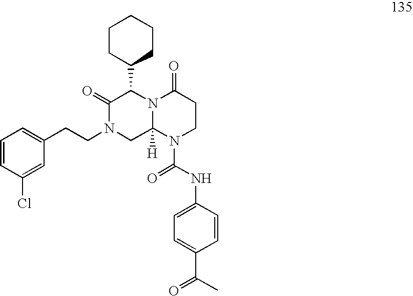

¹HNMR: (300 MHz, CDCl₃) δ 1.20 (m, 5H), 1.62 (m, 2H), 1.73 (m, 2H), 1.99 (m, 2H), 2.55 (m, 1H), 2.59 (s, 3H), 2.66 (m, 1H), 2.89 (m, 3H), 3.24 (m, 1H), 3.34 (m, 1H), 3.58 (m, 2H), 3.86 (m, 1H), 5.06 (d, 1H, J=6.95 Hz), 5.98 (dd, 1H, J=4.40, 10.98 Hz), 6.85 (br, s, 1H), 7.10 (m, 1H), 7.22 (m, 3H), 7.50 (d, 2H, J=8.42 Hz), 7.96 (d, 2H, J=8.42 Hz).

LC/MS: (method A) m/z 552.54, Rf 1.837, 89.8% purity.

### Example 52

### (6S,9aS)-6-Cyclohexyl-4,7-dioxo-8-(2-thiophen-2-yl-ethyl)-hexahydro-pyrazino[1,2-a]pyrimidine-1-carboxylic acid (4-acetyl-phenyl)-amide 138

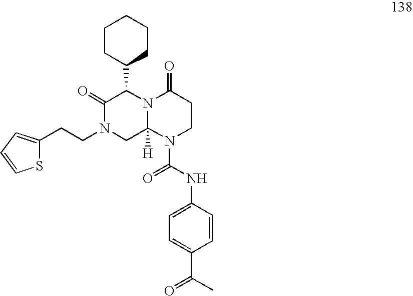

¹HNMR: (300 MHz, CDCl₃) δ 1.20 (m, 5H), 1.65 (m, 2H), 1.73 (m, 2H), 2.01 (m, 2H), 2.54 (m, 2H), 2.58 (s, 3H), 2.70 (m, 1H), 3.03-3.33 (br, m, 4H), 3.58 (m, 2H), 3.90 (m, 1H), 5.07 (d, 1H, J=6.96 Hz), 6.00 (dd, 1H, J=4.02, 10.25 Hz), 6.85 (m, 1H), 6.94 (m, 1H), 7.03 (br, s, 1H), 7.15 (d, 1H, J=5.12 Hz), 7.49 (d, 2H, J=8.42 Hz), 7.94 (d, 2H, J=8.42 Hz).

LC/MS: (method A) m/z 523.35, Rf 1.707, 90.6% purity.

### Example 53

### (6S,9aS)-6-Cyclohexyl-4,7-dioxo-8-(2-pyridin-3-yl-ethyl)-hexahydro-pyrazino[1,2-a]pyrimidine-1-carboxylic acid (4-acetyl-phenyl)-amide 140

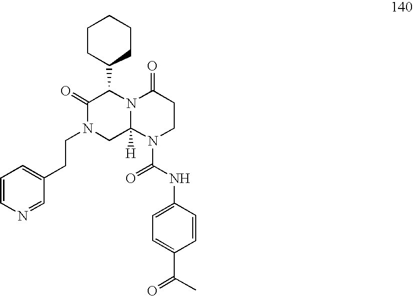

LC/MS: (method A) m/z 518.43, Rf 1.310, 97.0% purity.

### Example 54

### (6S,9aS)-6-Cyclohexylmethyl-4,7-dioxo-8-phenethyl-hexahydro-pyrazino[1,2-a]pyrimidine-1-carboxylic acid 4-acetyl-2-methyl-phenyl ester 144

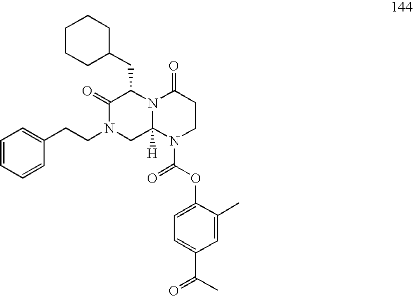

LC/MS: (method A) m/z 546.36, Rf 1.883, 96.1% purity.

### Example 55

### (6S,9aS)-6-Cyclohexylmethyl-4,7-dioxo-8-phenethyl-hexahydro-pyrazino[1,2-a]pyrimidine-1-carboxylic acid [4-(1-hydroxy-ethyl)-phenyl]-amide 145

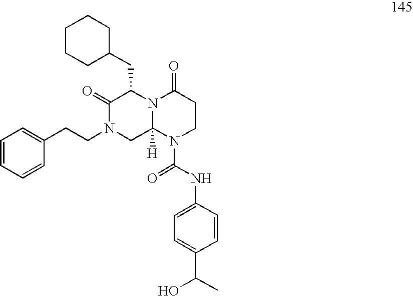

LC/MS: (method A) m/z 533.34, Rf 1.680, 91.4% purity.

### Example 56

### (6S,9aS)-6-Cyclohexylmethyl-4,7-dioxo-8-phenethyl-hexahydro-pyrazino[1,2-a]pyrimidine-1-carboxylic acid 4-acetyl-3-methyl-phenyl ester 146

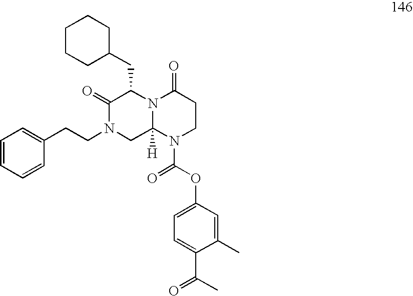

LC/MS: (method A) m/z 546.35, Rf 1.907, 72.0% purity.

### Example 57

### (6S,9aS)-6-Cyclohexyl-4,7-dioxo-8-phenethyl-hexahydro-pyrazino[1,2-a]pyrimidine-1-carboxylic acid 4-acetyl-3-methyl-phenyl ester 148

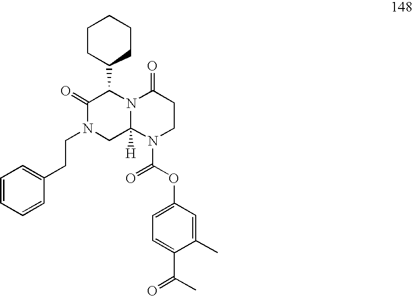

¹H NMR: (300 MHz, CDCl₃) δ 1.19 (m, 5H), 1.59 (m, 2H), 1.75 (m, 2H), 1.97 (m, 2H), 2.53 (m, 1H), 2.56 (s, 3H), 2.58 (s, 3H), 2.62 (m, 1H), 2.93 (m, 2H), 3.26 (m, 3H), 3.59 (m, 2H), 4.29 (m, 1H), 5.07 (d, 1H, J=6.59 Hz), 5.83 (dd, 1H, J=4.39, 10.25 Hz), 7.03 (m, 2H), 7.25 (m, 5H), 7.77 (d, 1H, J=8.42 Hz).

LC/MS: (method A) m/z 532.30, Rf 1.820, 98.6% purity.

### Example 58

### (6S,9aS)-6-Cyclohexyl-4,7-dioxo-8-phenethyl-hexahydro-pyrazino[1,2-a]pyrimidine-1-carboxylic acid 4-acetyl-phenyl ester 149

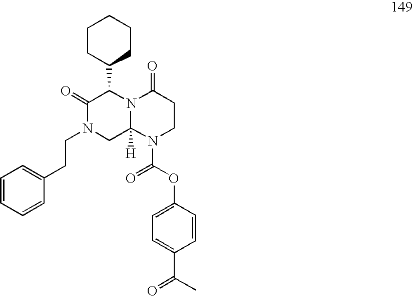

¹H NMR: (300 MHz, CDCl₃) δ 1.19 (m, 5H), 1.61 (m, 2H), 1.75 (m, 2H), 1.98 (m, 2H), 2.50 (m, 1H), 2.62 (s, 3H), 2.68 (m, 1H), 2.93 (m, 2H), 3.23 (m, 3H), 3.59 (m, 2H), 4.30 (m, 1H), 5.08 (d, 1H, J=6.59 Hz), 5.83 (dd, 1H, J=4.39, 10.25 Hz), 7.25 (m, 7H), 8.03 (d, 2H, J=8.05 Hz).

LC/MS: (method A) m/z 518.26, Rf 1.757, 84.8% purity.

### Example 59

### (6S,9aS)-6-Cyclohexyl-4,7-dioxo-8-phenethyl-hexahydro-pyrazino[1,2-a]pyrimidine-1-carboxylic acid 3-oxo-2,3-dihydro-benzofuran-6-yl ester 150.

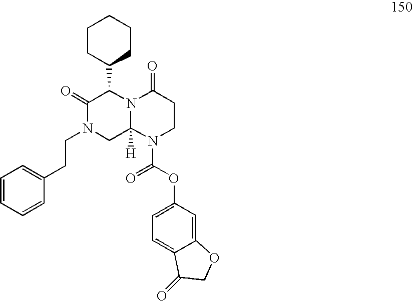

LC/MS: (method A) m/z 532.22, Rf 1.707, 89.0% purity.

### Example 60

### 6-(3,4-Difluoro-phenyl)-8-[2-(3-fluoro-phenyl)-ethyl]-4,7-dioxo-hexahydro-pyrazino [1,2-a]pyrimidine-1-carboxylic acid (4-acetyl-phenyl)-amide 151

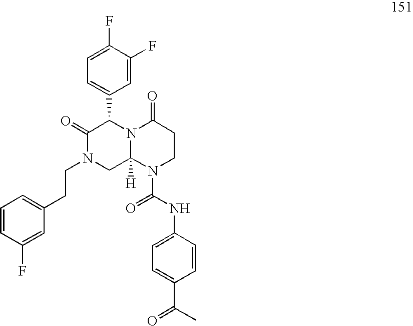

¹H NMR: (300 MHz, CDCl₃) δ2.53 (m, 1H), 2.55 (s, 3H), 2.69 (m, 1H), 2.95 (m, 2H), 3.33-3.55 (br, m, 3H), 3.67 (m, 2H), 4.06 (m, 1H), 5.79 (dd, 1H, J=4.02, 10.24 Hz), 6.34 (s, 1H), 6.97 (m, 5H), 7.20 (m, 1H), 7.45 (d, 2H, J=8.79 Hz), 7.89 (m, 3H), 7.98 (s, 1H).

LC/MS: (method A) m/z 565.16, Rf 1.693, 89.8% purity.

### Example 61

### (6S,9aS)-6-(3-Fluoro-phenyl)-8-[2-(3-fluoro-phenyl)-ethyl]-4,7-dioxo-hexahydro-pyrazino[1,2-a]pyrimidine-1-carboxylic acid (4-acetyl-phenyl)-amide 152

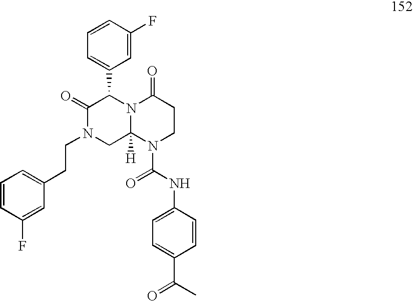

¹H NMR: (300 MHz, CDCl₃) δ 2.49 (m, 1H), 2.54 (s, 3H), 2.64 (m, 1H), 2.94 (m, 2H), 3.33-3.77 (br, m, 5H), 4.00 (m, 1H), 5.80 (dd, 1H, J=4.40, 10.24 Hz), 6.37 (s, 1H), 7.01 (m, 6H), 7.25 (m, 2H), 7.41 (d, 2H, J=8.42 Hz), 7.73 (br, s, 1H), 7.85 (d, 2H, J=8.42 Hz).

LC/MS: (method A) m/z 547.40, Rf 1.557, 93.1% purity.

### Example 62

### (6S,9aS)-6-(2,4-Difluoro-phenyl)-8-[2-(3-fluoro-phenyl)-ethyl]-4,7-dioxo-hexahydro-pyrazino[1,2-a]pyrimidine-1-carboxylic acid (4-acetyl-phenyl)-amide 153

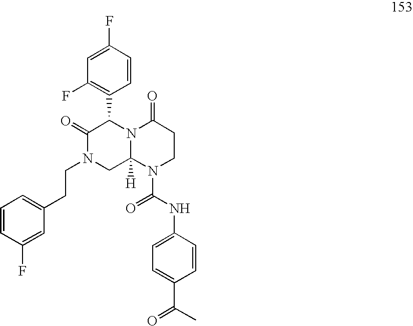

¹H NMR: (300 MHz, CDCl₃) δ 2.58 (m, 5H), 2.95 (m, 2H), 3.41-3.67 (br, m, 4H), 3.74 (m, 1H), 3.90 (m, 1H), 6.10 (m, 1H), 6.35 (s, 1H), 6.83 (m, 2H), 6.94 (m, 2H), 7.02 (m, 1H), 7.09 (br, s, 1H), 7.30 (m, 1H), 7.41 (m, 3H), 7.91 (d, 2H, J=8.78 Hz).

LC/MS: (method A) m/z 565.23, Rf 1.607, 84.1% purity.

### Example 63

### (6S,9aS)-6-(2,3-Difluoro-phenyl)-8-[2-(3-fluoro-phenyl)-ethyl]-4,7-dioxo-hexahydro-pyrazino[1,2-a]pyrimidine-1-carboxylic acid (4-acetyl-phenyl)-amide 154

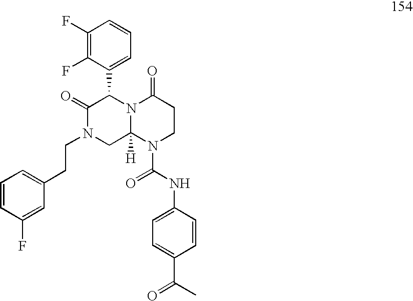

¹H NMR: (300 MHz, CDCl₃) δ 2.42 (m, 1H), 2.53 (s, 3H), 2.89 (m, 2H), 3.38-3.77 (br, m, 6H), 4.00 (m, 1H), 6.06 (dd, 1H, J=4.03, 10.25 Hz), 6.38 (s, 1H), 6.89-7.15 (br, m, 6H), 7.42 (d, 2H, J=8.42 Hz), 7.85 (m, 3H), 8.07 (br, s, 1H).

LC/MS: (method A) m/z 565.43, Rf 1.803, 95.6% purity.

### Example 64

### 6-(2,3-Difluoro-phenyl)-8-[2-(3-fluoro-phenyl)-ethyl]-4,7-dioxo-hexahydro-pyrazino[1,2-a]pyrimidine-1-carboxylic acid (4-acetyl-phenyl)-amide 155

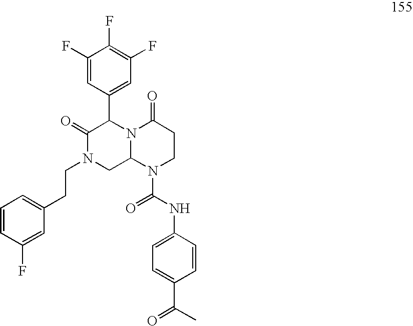

LC/MS: (method A) m/z 583.10, Rf 1.947, 94.4% purity.

### In Vitro Study: Reduction in CGRP-Induced Stimulation of cAMP

The CGRP receptor has been shown to be coupled to the Gs protein, the stimulatory subunit of G-protein which in turn activates adenyl cyclase, causing an increase in cAMP. Certain test compounds of the present invention were tested in a functional assay as described in TiPS (21): 432-438 (2000) that measures the production of cAMP when stimulated by CGRP. Assays were performed with SK-N-MC cells that endogenously express the human CGRP receptor. Cells were pre-incubated at various concentrations of test compounds for 15 minutes before exposure 3 nM CGRP. The accumulation of cAMP was measured after 30 minutes using the cAMP SPA Direct Screening Assay (Amersham). Selected test compounds were found to be antagonists as they displayed dose dependent blockage in CGRP-induced cAMP stimulation.

Tissue culture. SK-N-MC cells were grown at 37° C. in 5% CO₂ as a monolayer in medium consisting of MEM with Earle's salts and L-glutamine (Gibco) supplemented with 10% fetal bovine serum (Gibco).

Radioligand binding assays. Cells were rinsed twice with phosphate-buffered saline (155 mM NaCl, 3.3 mM Na₂HPO₄, 1.1 mM KH₂PO₄, pH 7.4), and incubated for 5-10 min. at 4° C. in hypotonic lysis buffer consisting of 10 mM Tris (pH 7.4) and 5 mM EDTA. Cells were transferred from plates to polypropylene tubes (16×100 mm), homogenized and centrifuged at 32,000×g for 30 min. Pellets were resuspended in buffer consisting of 50 mM Tris (pH 7.4) and 1 mM EDTA, homogenized, and centrifuged again at 32,000×g for 30 min. Pellets were resuspended in 100 mM Tris-Cl pH 7.5, 10 mM MgCl₂, 0.1% mammalian protease inhibitor cocktail (Sigma) and stored at −80° C. until needed. On the day of an experiment, homogenates were thawed and homogenized again. Binding of [¹²⁵I]-CGRP (NEN) was carried out in buffer containing 50 mM Tris-Cl pH 7.5, 5 mM MgCl₂, 0.5% BSA. Homogenates (3.5 μg protein/well) were incubated with competitor compounds (solubilized in 100% DMSO at 100× their final assay concentrations) for one hour at room temperature. Then [¹²⁵I]-CGRP was added (40 pM final concentration) and the reaction incubated for one additional hour at room temperature. Assays were stopped by addition of cold wash buffer (20 mM Tris-Cl pH 7.5, 0.1% BSA) immediately followed by filtration over glass fiber filters (Whatman GF/B) previously soaked in 0.5% PEI. Non-specific binding was defined with 100 nM CGRP.

By way of example, the results obtained for some of the test compounds are given in the table below:

Binding Data:

| Compound Number | IC₅₀ (nM) |
|---|---|
| 1 | 80 |
| 17 | 417 |
| 18 | 293 |
| 19 | 8000 |
| 20 | 907 |
| 21 | 233 |
| 22 | 2194 |
| 23 | 184 |
| 24 | 213 |
| 25 | 30000 |
| 26 | 117 |
| 27 | 1326 |
| 28 | 188 |
| 29 | 5161 |
| 30 | 2207 |
| 31 | 1052 |
| 32 | 598 |
| 33 | 2255 |
| 34 | 1173 |
| 35 | 244 |
| 36 | 350 |
| 37 | 704 |
| 39 | 233 |
| 40 | 2364 |
| 41 | 237 |
| 42 | 778 |
| 43 | 4000 |
| 44 | 3441 |
| 45 | 1945 |
| 46 | 167 |
| 47 | 192 |
| 48 | 115 |
| 52 | 358 |
| 53 | 2974 |
| 54 | 1267 |
| 55 | 400 |
| 56 | 665 |
| 57 | 500 |
| 58 | 814 |
| 59 | 271 |
| 60 | 265 |
| 61 | 564 |
| 62 | 624 |
| 63 | 3043 |
| 64 | 473 |
| 65 | 517 |
| 111 | 12000 |
| 116 | 2580 |
| 122 | 890 |
| 132 | 8000 |
| 135 | 7071 |
| 138 | 87 |
| 140 | 2440 |
| 144 | 7402 |
| 145 | 2730 |
| 146 | 364 |
| 148 | 3900 |
| 149 | 8560 |
| 150 | 1728 |
| 151 | 39 |
| 152 | 159 |
| 153 | 320 |
| 154 | 363 |
| 155 | 36 |

1. A compound of Formula I or a pharmaceutically acceptable salt or solvate thereof 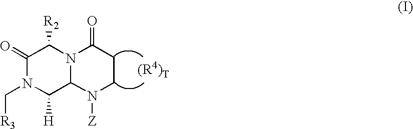
wherein
Z is CONHR₁ or CO₂R₁ wherein
R₁ is phenyl substituted in the para position with a ketone or isostere thereof or an alcohol or isostere thereof;
R₂ is
C₁-C₄ alkyl, C₃-C₈ cycloalkyl, acetic acid adamantan-2-yl ester, acetic acid benzyl ester, butyl carbamic acid benzyl ester, benzyloxy methyl, 4-benzyloxy benzyl, piperidin-4-yl, tetrahydro-pyran-4-yl, 3-methyl-3H-imidazol-4-yl methyl, 2-carbamoyl-ethyl, C₁-C₄ alkyl S(O)ₙ C₁-C₄ alkyl wherein n is 0, 1, or 2, benzyl or phenyl, wherein either or both of said benzyl or phenyl are optionally substituted on the aryl moiety with one or more of the same or different substituents selected from the group consisting of
S(O)ₘ C₁-C₄ alkyl wherein m is 0, 1 or 2;
nitro;
hydroxy;
fluoro; and
chloro;
R₃ is
benzyl optionally substituted in the meta position with fluoro or chloro,
2-pyridin-4-yl-methyl, 2-thiophen-2-yl methyl or 2-pyridin-3-yl-methyl;
R₄ together with the carbon atoms to which it is attached is phenyl or napthyl; and
T is 0 or 1.
